(19) United States
(12) Patent Application Publication
(10) Pub. No.: US 2009/0074692 A1
(43) Pub. Date: Mar. 19, 2009

(54) COSMETIC COMPOSITION COMPRISING AT LEAST ONE CATIONIC COPOLYMER AND AT LEAST ONE ANIONIC ASSOCIATIVE POLYMER AND COSMETIC TREATMENT PROCESSES USING SAID COMPOSITION

(76) Inventors: Olga BIGANSKA, Asnieres sur Seine (FR); Pascale Lazzeri-Vigouroux, Puyricard (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/210,713

(22) Filed: Sep. 15, 2008

Related U.S. Application Data
(60) Provisional application No. 60/960,231, filed on Sep. 21, 2007.

(30) Foreign Application Priority Data
Sep. 14, 2007 (FR) 07/57582

(51) Int. Cl. A61K 8/81; A61K 8/88; A61Q 5/12

(52) U.S. Cl. 424/70.2; 424/70.16; 424/70.17

(57) ABSTRACT

The disclosure relates to novel cosmetic compositions comprising, in a cosmetically acceptable medium:
(i) at least one cationic polymer which are produced by polymerization of a mixture of monomers comprising:
   a) at least one vinyl monomer substituted with at least one amino group,
   b) at least one hydrophobic nonionic vinyl monomer,
   c) at least one associative vinyl monomer, and
   e) at least one hydroxylated nonionic vinyl monomer, and
(ii) at least one anionic associative polymers.

This application claims benefit of U.S. Provisional Application No. 60/960,231, filed Sep. 21, 2007, the contents of which are incorporated herein by reference. This application also claims benefit of priority under 35 U.S.C. § 119 to French Patent Application No. FR 0757582, filed Sep. 14, 2007, the contents of which are also incorporated herein by reference.

The present disclosure relates to a cosmetic composition, such as for conditioning hair, comprising at least one anionic associative polymer and at least one cationic copolymer. The present disclosure also relates to a cosmetic treatment process for keratin materials, such as hair.

Hair compositions, such as a shampoo or a conditioner, are most commonly in the form of a liquid having different viscosities. Products of which the texture is sufficiently thick to remain on the hair, without running, for a certain period of time, are desirable. This thicker, or even gel, texture, should not impair the qualities of the product when used.

The thickening and/or gelling of aqueous media with polymers has been the focus of cosmetic research in the cosmetic and pharmaceutical fields for some time. Obtaining an advantageous thickening effect with a water-soluble polymer generally correlates to a high molar mass and a large hydrodynamic volume. The gelling of an aqueous medium is a result of a three-dimensional polymer network obtained by crosslinking of linear polymers or by copolymerization of bifunctional and polyfunctional monomers. However, the use of such polymers having very high molar masses creates problems, such as relatively unpleasant texture and poor spreadability of the gels obtained.

One approach to overcome those problems used thickener polymers capable of reversibly associating with each other or with other molecules or particles. This physical association gives rise to thixotropic or shear-thinning macromolecular systems, such as systems whose viscosity depends on the shear forces to which they are subjected.

Such polymers capable of reversibly associating with each other or with other molecules are known as associative polymers. The interaction forces involved may be of very different natures, for example electrostatic, hydrogen bonding, or hydrophobic interactions.

One class of associative polymers is amphiphilic polymers, such as polymers comprising at least one hydrophilic part that makes them water-soluble and at least one hydrophobic region via which the polymers interact and assemble with each other or with other molecules.

The use of associative polymers, such as anionic associative polymers, in cosmetics has been previously recommended. However, the rheological and cosmetic properties of such polymers are not optimal.

Furthermore, such compositions have drawbacks, such as problems with rinseability, stability, and syneresis; they are difficult to distribute over keratin materials, and they also exhibit insufficient cosmetic properties.

In summary, current cosmetic conditioning compositions comprising anionic associative polymers are not entirely satisfactory. Thus, it is desirable to obtain cosmetic compositions having a pleasant texture, a viscosity that is sufficient to prevent running, and good cosmetic properties, such as for use on very sensitized hair. Furthermore, compositions of the present disclosure must be stable during storage.

The applicant has now discovered that a cosmetic composition comprising at least one anionic associative polymer and at least one cationic copolymer remedies the drawbacks discussed above.

The compositions according to the present disclosure give hair more mass, more body, and more sheen compared with compositions comprising an anionic associative polymer in the absence of at least one cationic copolymer.

Compositions of the present disclosure have a texture which remains the same or changes only slightly over time at ambient temperature (approximately 25° C.) and at 45° C. The stability of these compositions is therefore satisfactory at ambient temperature and 45° C. These compositions allow for a better distribution of the product during application. When the compositions further comprise foaming surfactants, their lathering properties, at, for example, the start of lathering, are improved.

Thus, the present disclosure provides novel cosmetic compositions comprising, in a cosmetically acceptable medium:
(i) at least one cationic polymer which is produced by polymerization of a mixture of monomers comprising:
   a) at least one vinyl monomer substituted with at least one amino group,
   b) at least one hydrophobic nonionic vinyl monomer, chosen from a compound of formula (I) and (II):

CH₂═C(X)Z, (1)

CH₂═CH—OC(O)R; (II)
wherein in the formulae (I) and (II):
X is chosen from a hydrogen atom and a methyl group;
Z is chosen from —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R is independently a C₁-C₃₀ alkyl group;
each R¹ is independently chosen from a C₁-C₃₀ alkyl group, a hydroxylated C₂-C₃₀ alkyl group, and a halogenated C₁-C₃₀ alkyl group;
c) at least one associative vinyl monomer, and
e) at least one hydroxylated nonionic vinyl monomer; and
(ii) at least one anionic associative polymer.

Another embodiment of the present disclosure comprises a cosmetic treatment process for keratin materials, such as hair, using the above mentioned composition.

Another embodiment of the present disclosure is the use of said composition as a conditioner or a shampoo.

Based on the present specification and examples recited below, one of skill in the art will recognize other exemplary embodiments of the present disclosure.

The term “sensitized hair” means, according to the present disclosure, hair that has undergone outside physical attacks (light, heat, waves, etc.), mechanical attacks (repeated blow-drying, combing or brushing, etc.), or chemical attacks (oxidation dyeing, bleaching, permanent-waving, hair straightening, etc.).

The term “cosmetically acceptable medium” means a medium compatible with all keratin materials, such as skin, hair, nails, eyelashes, eyebrows, lips, and any other area of the body or the face.

One feature of the present disclosure is the presence of at least one cationic polymer, which is obtained by polymerization of a mixture of monomers comprising a) at least one vinyl monomer substituted with at least one amine group, b) at least one hydrophobic nonionic vinyl monomer, c) at least one associative vinyl monomer, and e) at least one hydroxylated nonionic vinyl monomer. The monomers constituting the at least one cationic copolymer may also comprise at least one semi-hydrophobic surfactant vinyl monomer d). The monomers a) to e) are different from one another.

The at least one cationic polymer (i) may be a thickening polymer.

For the purpose of the present disclosure, the term “thickening polymer” means a polymer which, when introduced at 1% by weight into an aqueous or aqueous-alcoholic solution containing 30% by weight of ethanol, and at pH 7, makes it possible to attain a viscosity of at least 100 cps at 25° C., and a shear rate of 1 s⁻¹. The viscosity can be measured using a viscometer with cone-plate geometry, for example a Haake RS 600 rheometer. These polymers make it possible to increase the viscosity of the compositions which contain them by at least 50 cps at 25° C. and at 1 s⁻¹.

The at least one cationic polymer (i) used in the composition according to the present disclosure, and the process for producing it, are described, for example, in international application WO 2004/024779.

For the purpose of the present disclosure, the term “vinyl monomer” means a monomer comprising at least one R₀CH═C(R₀)— group, wherein each R₀ is independently chosen from a hydrogen atom, a C₁-C₃₀ alkyl, —C(O)OH or C(O)OR₁₀, —O—C(O)OR₁₀, —C(O)NHR₁₀, and C(O)NR′₀R′₀, R′₀ and R″₀, which may be identical or different, are C₁-C₃₀ alkyl groups.

Thus, for example, for the purpose of the present disclosure, (meth)acrylates and (meth)acrylamides are vinyl monomers.

As previously explained, the mixture of monomers for the preparation of the at least one cationic polymer (i) used in the composition according to the present disclosure comprises at least one vinyl monomer substituted with at least one amino group.

The at least one vinyl monomer substituted with at least one amino group that can be used for the preparation of the at least one cationic polymer used according to the present disclosure are basic and polymerizable ethylenically unsaturated monomers. The at least one amine group may derive from mono-, di-, or polyaminated alkyl groups, or from heteroaromatic groups containing a nitrogen atom. The at least one amine group may be a primary, secondary, or tertiary amine. These monomers may be used in the amine form or in the salt form.

In one aspect of the disclosure, the at least one vinyl monomer substituted with at least one amine group is chosen from:

mono(C₁-C₄)alkylamino(C₁-C₈)alkyl (meth)acrylates,

di(C₁-C₄)alkylamino(C₁-C₈)alkyl (meth)acrylates, preferably di(C₁-C₄)alkylamino(C₁-C₆)alkyl (meth)acrylates,

mono(C₁-C₄)alkylamino(C₁-C₈)alkyl(meth)acrylamides,

di(C₁-C₄)alkylamino(C₁-C₈)alkyl(meth)acrylamides,

(meth)acrylamides comprising a heterocyclic group, or group containing a nitrogen atom,
(meth)acrylates comprising a heterocyclic group, or group containing a nitrogen atom, and

nitrogenous heterocycles comprising a vinyl group.

Non-limiting examples of vinyl monomers substituted with at least one amino group include

mono- and di(C₁-C₄ alkyl)amino(C₁-C₄ alkyl) (meth)acrylates, such as 2-(N,N-dimethylamino)ethyl (meth)acrylate, 3-(N,N-dimethylamino)propyl (meth)acrylate, 4-(N,N-dimethylamino)butyl (meth)acrylate, (N,N-dimethylamino)-t-butyl (meth)acrylate, 2-(N,N-diethylamino)ethyl (meth)acrylate, 3-(N,N-diethylamino)propyl (meth)acrylate, 4-(N,N-diethylamino)butyl (meth)acrylate, 2-(N,N-dipropylamino)ethyl (meth)acrylate, 3-(N,N-dipropylamino)propyl (meth)acrylate and 4-(N,N-dipropylamino)butyl (meth)acrylate;

mono- and di(C₁-C₄ alkyl)amino(C₁-C₄ alkyl)(meth)acrylamides, such as N′-(2-N,N-dimethylamino)ethyl(meth)acrylamide and N′-(3-N,N-dimethylamino)propylacrylamide;

(meth)acrylamides and (meth)acrylates comprising a heterocyclic group containing a nitrogen atom, such as N-(2-pyridyl)acrylamide, N-(2-imidazolyl)methacrylamide, 2-(4-morpholinyl)ethyl methacrylate, 2-(4-morpholinyl)ethyl acrylate, N-(4-morpholinyl)methacrylamide and N-(4-morpholinyl)acrylamide; and

nitrogenous heterocycles comprising a vinyl group or groups, such as 2-vinylpyridine and 4-vinylpyridine.

When the monomers are in the form of salts, they may be mineral salts, such as hydrochloride, sulfate, and phosphate salts, or else salts of organic acids, such as acetate, maleate, and fumarate salts.

Non-limiting exemplary vinyl monomers substituted with at least one amino group include:
3-(N,N-dimethylamino)propyl (meth)acrylate,
N′-(3-N,N-dimethylamino)propyl(meth)acrylamide,
2-(N,N-dimethylamino)ethyl (meth)acrylate,
2-(N,N-diethylamino)ethyl (meth)acrylate,
2-(tert-butylamino)ethyl (meth)acrylate,
2-(N,N-dimethylamino)propyl(meth)acrylamide, and
2-(N,N-dimethylamino)neopentyl acrylate.

The at least one vinyl monomer substituted with at least one amino group is present in an amount ranging from 10% to 70% by weight, such as from 20% to 60% by weight, such as from 30% to 40% by weight, relative to the total weight of the mixture of monomers.

As previously explained, the mixture of monomers for the preparation of the at least one cationic polymer (i) used according to the present disclosure also comprises at least one hydrophobic nonionic vinyl monomer b).

The at least one hydrophobic nonionic vinyl monomer that can be used for the preparation of the at least one cationic polymer used according to the present disclosure is chosen from a compound of formula (I) and (II):

CH₂═C(X)Z, (I)

CH₂═CH—OC(O)R; (II)

wherein in the formulae (I) and (II):

X is chosen from a hydrogen atom or a methyl group;

Z is chosen from —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;

x is an integer ranging from 1 to 6;

each R is independently a C₁-C₃₀ alkyl group; and

each R¹ is independently chosen from a C₁-C₃₀ alkyl group, a hydroxylated C₂
C₃₀ alkyl group, and a halogenated C₁-C₃₀ alkyl group.

Non-limiting mention may be made of C₁-C₃₀ alkyl (meth)acrylates; (C₁-C₃₀ alkyl)(meth)acrylamides; styrene, substituted styrenes, such as vinyltoluene (or 2-methylstyrene), butylstyrene, isopropylstyrene, para-chlorostyrene; vinyl esters, such as vinyl acetate, vinyl butyrate, vinyl caprolate, vinyl pivalate, and vinyl neodecanoate; unsaturated nitriles, such as (meth)acrylonitrile and acrylonitrile; and unsaturated silanes, such as trimethylvinylsilane, dimethylethylvinylsilane, allyldimethylphenylsilane, allyltrimethylsilane, 3-acrylamidopropyltrimethylsilane, and 3-trimethylsilylpropyl methacrylate.

In one embodiment, the at least one hydrophobic nonionic vinyl monomer is chosen from C₁-C₃₀ alkyl acrylates, and C₁-C₃₀ alkyl methacrylates, such as ethyl acrylate, methyl methacrylate, and 3,3,5-trimethylcyclohexyl methacrylate.

The at least one hydrophobic nonionic vinyl monomer is present in an amount ranging from 20% to 80% by weight, such as from 20% to 70% by weight, and such as from 50% to 65% by weight, relative to the total weight of the mixture of monomers.

The at least one associative vinyl monomer that can be used for the preparation of the at least one cationic polymer (i) used according to the present disclosure is chosen from compounds having an end (i)′ comprising ethylenic unsaturation(s) for addition polymerization with other monomers of the system; a central polyoxyalkylene portion (ii)′ for conferring selective hydrophilic properties on the polymers; and a hydrophobic end (iii)′ for conferring selective hydrophobic properties on the polymers.

The end (i)′ comprising ethylenic unsaturation(s) of the at least one associative vinyl monomer is derived from a monocarboxylic or dicarboxylic acid or anhydride comprising α, β-ethylenic unsaturation, such as a C₃ or C₄ monocarboxylic or dicarboxylic acid or anhydride. Alternatively, the end (i)′ of the at least one associative monomer may be derived from an allyl ether or from a vinyl ether; from a nonionic urethane monomer substituted with a vinyl group, as disclosed in U.S. Pat. No. RE33, 156 or in U.S. Pat. No. 5,294,692; or from a urea reaction product substituted with a vinyl group, as disclosed in U.S. Pat. No. 5,011,978.

The central portion (ii)′ of the at least one associative vinyl monomer may be a polyoxyalkylene segment comprising from 5 to 250, such as from 10 to 120, and such as from 15 to 60 C₂-C₇ alkylene oxide units. Central portions (ii)′ may be polyoxyethylene, polyoxypropylene, and polyoxybutylene segments comprising from 5 to 150, such as from 10 to 100, and such as from 15 to 60 ethylene oxide, propylene oxide, or butylene oxide units, and random or nonrandom blocks of ethylene oxide, propylene oxide, or butylene oxide units. In at least one embodiment, the central portions are polyoxyethylene segments.

The hydrophobic end (iii)′ of the at least one associative monomer is a hydrocarbon-based fragment belonging to one of the following hydrocarbon-based categories: a linear C₈-C₄₀ alkyl, a C₂-C₄₀ alkyl substituted with an aryl group, a phenyl substituted with a C₂-C₄₀ alkyl group, a branched C₈-C₄₀ alkyl, a C₈-C₄₀ alicyclic group, and a C₈-C₈₀ complex ester.

For the purpose of the present disclosure, the term “complex ester” means any ester other than a simple ester.

For the purpose of the present disclosure, the term “simple ester” means any unsubstituted, linear or branched, C₁-C₃₀ saturated aliphatic alcohol ester.

Non-limiting examples of hydrophobic ends (iii)′ of the at least one associative monomer include linear or branched alkyl groups containing from 8 to 40 carbon atoms, such as (C₈) capryl, (branched C₈) isooctyl, (C₁₀) decyl, (C₁₂) lauryl, (C₁₄) myristyl, (C₁₆) cetyl, (C₁₆-C₁₈) cetearyl, (C₁₈) stearyl, (branched C₁₈) isostearyl, (C₂₀) arachidyl, (C₂₂) behenyl, (C₂₄) lignoceryl, (C₂₆) cerotyl, (C₂₈) montanyl, (C₃₀) melissyl, and (C₃₂) lacceryl.

Non-limiting examples of linear or branched alkyl groups containing from 8 to 40 carbon atoms and derived from a natural source are alkyl groups derived from hydrogenated groundnut oil, from soybean oil, and from canola oil (predominantly C₁₈), hydrogenated C₁₆-C₁₈ tallow oil, and hydrogenated C₁₀-C₃₀ terpenols, such as hydrogenated geraniol (branched C₁₀), hydrogenated farnesol (branched C₁₅), and hydrogenated phytol (branched C₂₀).

Non-limiting examples of phenyl groups substituted with a C₂-C₄₀ alkyl include octylphenyl, nonylphenyl, decylphenyl, dodecylphenyl, hexadecylphenyl, octadecylphenyl, isooctylphenyl, and sec-butylphenyl.

C₈-C₄₀ alicyclic groups may, for example, be groups derived from sterols of animal origin, such as cholesterol, lanosterol, or 7-dehydrocholesterol; or else derivatives of plant origin, such as phytosterol, stigmasterol, or campesterol; or else derivatives derived from microorganisms, such as ergosterol or mycrosterol. Other C₈-C₄₀ alicyclic groups that can be used in the present disclosure are, for example, cyclooctyl, cyclododecyl, adamantyl, and decahydronaphthyl, and groups derived from natural C₈-C₄₀ alicyclic compounds such as pinene, hydrogenated retinol, camphor, and isobornyl alcohol.

The C₂-C₄₀ alkyl groups substituted with an aryl group may, for example, be 2-phenylethyl, 2,4-diphenylbutyl, 2,4,6-triphenylhexyl, 4-phenylbutyl, 2-methyl-2-phenylethyl, or 2,4,6-tri(1′-phenylethyl)phenyl.

Non-limiting examples of C₈-C₄₀ complex esters that can be used as end (iii)′ include hydrogenated castor oil (principally 12-hydroxystearic acid triglyceride); 1,2-diacyiglycerols, such as 1,2-distearyl glycerol, 1,2-dipalmityl glycerol, or 1,2-dimyristyl glycerol; di-, tri- or polyesters of sugars, such as 3,4,6-tristearyl glucose or 2,3-dilauryl fructose; and sorbitan esters such as those disclosed in U.S. Pat. No. 4,600,761.

The at least one associative vinyl monomer that can be used according to the present disclosure may be prepared by any method known from the prior art, such as, for example, as disclosed in U.S. Pat. Nos. 4,421,902; 4,384,096; 4,514,552; 4,600,761; 4,616,074; 5,294,692; 5,292,843; 5,770,760; and 5,412,142.

The at least one associative vinyl monomer c) that can be used according to the present disclosure is chosen from a compound of formula (III):

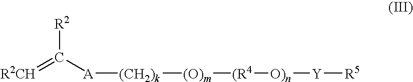

wherein:
each R² is independently chosen from a hydrogen atom, a methyl group, a —C(O)OH group, and a —C(O)OR³ group;

R³ is a C₁-C₃₀ alkyl;

A is chosen from a —CH₂C(O)O—, —C(O)O—, —O—, CH₂O, —NHC(O)NH—, —C(O)NH—, —Ar—(CE₂)_{z}—NHC(O)O—, —Ar—(CE₂)_{z}—NHC(O)NH—, and —CH₂CH₂—NHC(O)— group;

Ar is a divalent aryl group;

E is chosen from a hydrogen atom and a methyl group;

z is equal to 0 or 1;

k is an integer ranging from 0 to 30;

m is equal to 0 or 1, with the proviso that, when k=0, m=0, and when k ranges from 1 to 30, m is equal to 1;

(R⁴—O)ₙ is a polyoxyalkylene, which is a homopolymer, a random copolymer, or a block copolymer, with C₂-C₄ oxyalkylene units;

R⁴ is chosen from C₂H₄, C₃H₆, and C₄H₈, or mixtures thereof;

n is an integer ranging from 5 to 250;

Y is chosen from —R⁴⁰—, —R⁴NH—, —C(O)—, —C(O)NH—, R⁴NHC(O)NH—, and —C(O)NHC(O)—; and

R⁵ is a substituted or unsubstituted alkyl chosen from linear C₈-C₄₀ alkyls, branched C₈-C₄₀ alkyls, C₈-C₄₀ alicyclics, phenyls substituted with a C₂-C₄₀ alkyl group, C₂-C₄₀ alkyls substituted with an aryl group, and C₈-C₈₀ complex esters;

the R⁵ alkyl group optionally comprising at least one substituent chosen from hydroxyl, alkoxy, and halo groups.

Non-limiting examples of the at least one associative vinyl monomer include polyethoxylated cetyl (meth)acrylates, polyethoxylated cetearyl (meth)acrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-tri(1′-phenylethyl)phenyl (meth)acrylates, polyethoxylated (meth)acrylates of hydrogenated castor oil, polyethoxylated canola (meth)acrylates, and polyethoxylated (meth)acrylates of cholesterol, where the polyethoxylated portion of the monomer comprises from 5 to 100, such as from 10 to 80, and such as from 15 to 60 ethylene oxide units.

Non-limiting examples of the at least one associative vinyl monomer include polyethoxylated cetyl methacrylates, polyethoxylated cetearyl methacrylates, polyethoxylated stearyl (meth)acrylates, polyethoxylated arachidyl (meth)acrylates, polyethoxylated behenyl (meth)acrylates, and polyethoxylated lauryl (meth)acrylates, where the polyethoxylated portion of the monomer comprises from 10 to 80, such as from 15 to 60, and such as from 20 to 40 ethylene oxide units.

In at least one embodiment, the at least one associative vinyl monomer is present in an amount ranging from 0.001% to 25% by weight, such as from 0.01% to 15% by weight, and such as from 0.1% to 10%, by weight of the mixture of monomers.

The at least one semi-hydrophobic surfactant vinyl monomer, optionally present in the mixture of monomers, can moderate the associative properties of the cationic associative polymers which contain them, thus producing aqueous gels having very good texture and rheological properties.

For the purpose of the present disclosure, the term “semi-hydrophobic surfactant vinyl monomer” means a monomer which has a structure similar to an associative monomer, but has a substantially nonhydrophobic end and thus does not confer an associative property on the polymers.

The associativity property of a polymer is linked to the property, in a given medium, of the molecules of said polymer of associating with one another, or of associating with molecules of a coagent, such as a surfactant coagent, which, within a certain concentration range, is reflected by an increase in the viscosity of the medium.

The at least one semi-hydrophobic surfactant vinyl monomer is, for example, a compound having two parts:

(i) an unsaturated end group for allowing addition polymerization with the other monomers of the reaction mixture, and

(ii) a polyoxyalkylene group for reducing the associations between the hydrophobic groups of the polymer or the hydrophobic groups of the other materials possibly present in the composition containing the polymer.

The end providing the vinyl or ethylenic unsaturation for the addition polymerization can be derived from an α,β-ethylenically unsaturated monocarboxylic or dicarboxylic acid or anhydride, such as a C₃-C₄ monocarboxylic or dicarboxylic acid or an anhydride of this acid. In another embodiment, the end (A) may derive from an allyl ether, a vinyl ether, or a urethane which is unsaturated and nonionic.

The polymerizable unsaturated end (A) may also derive from a C₈-C₃₀ unsaturated fatty acid containing at least one free carboxyl functional group. This C₈-C₃₀ group is part of the unsaturated end (A) and is different from the pendant hydrophobic groups of the associative monomers, which are separated from the unsaturated end of the associative monomer by a hydrophilic spacer group.

The polyoxyalkylene portion (B) comprises a long-chain polyoxyalkylene segment, which is similar to the hydrophilic portion of the associative monomers. Exemplary polyoxyalkylene portions (B) include C₂-C₄ polyoxyethylene, polyoxypropylene, and polyoxybutylene units comprising from 5 to 250, such as from 10 to 100 oxyalkylene units. When the at least one semi-hydrophobic surfactant vinyl monomer comprises more than one type of oxyalkylene unit, these units may be arranged in random or nonrandom sequences or in blocks.

In at least one embodiment, the at least one semi-hydrophobic surfactant vinyl monomer is chosen from compounds of formula (IV) and (V):

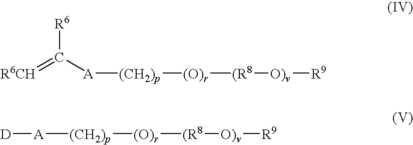

wherein, in each formula (IV) and (V),
each R⁶ is independently chosen from a hydrogen atom, a C₁-C₃₀ alkyl, —C(O)OH, and C(O)OR⁷;

R⁷ is a C₁-C₃₀ alkyl;

A is chosen from a —CH₂C(O)O—, —C(O)O—, —O—, —CH₂O, —NHC(O)NH—, —C(O)NH—, —Ar—(CE₂)_{z}—NHC(O)O—, —Ar—(CE₂)_{z}—NHC(O)NH—, and —CH₂CH₂NHC(O)— groups;

Ar is a divalent aryl group;

E is a hydrogen atom or a methyl group;

z is equal to 0 or 1;

p is an integer ranging from 0 to 30;

r is equal to 0 or 1, with the proviso that, when p is equal to 0, r is equal to 0, and when p ranges from 1 to 30, r is equal to 1;

(R₈—O)ᵥ is a polyoxyalkylene which is a homopolymer, a random copolymer, or a block copolymer with C₂-C₄ oxyalkylene units, wherein R⁸ is chosen from C₂H₄, C₃H₆, and C₄H₈, and v is an integer ranging from 5 to 250;

R⁹ is a hydrogen atom or a C₁-C₄ alkyl; and

D is an unsaturated C₈-C₃₀ alkyl or an unsaturated C₈-C₃₀ alkenyl substituted with a carboxyl group.

In at least one embodiment, the mixture of monomers comprises at least one semi-hydrophobic surfactant vinyl monomer chosen from a compound of the following formulae:

CH₂═CH—O(CH₂)ₐO(C₃H₆O)_{b}(C₂H₄O)_{c}H and

CH₂═CHCH₂O(C₃H₆O)_{d}(C₂H₄O)ₙH;

wherein

a is an integer ranging from 2 to 4;

b is an integer ranging from 1 to 10;

c is an integer ranging from 5 to 50;

d is an integer ranging from 1 to 10; and

e is an integer ranging from 5 to 50.

Non-limiting examples of semi-hydrophobic surfactant vinyl monomers include the polymerizable emulsifiers sold under the references Emulsogen® RP109, R208, R307, RAL109, RAL208 and RAL307 by the company Clariant; BX-AA-E5P5 sold by the company Bimax; and Maxemul® 5010 and 5011 sold by the company Uniqema. In at least one embodiment, the monomers are Emulsogen® R208, R307 and RAL307.

According to the manufacturers:

Emulsogen® R109 is a random ethoxylated/propoxylated 1,4-butanediol vinyl ether having the empirical formula:

CH₂═CH—O(CH₂)₄O(C₃H₆O)₄(C₂H₄O)₁₀H;

Emulsogen® R208 which is a random ethoxylated/propoxylated 1,4-butanediol vinyl ether having the empirical formula:

CH₂═CH—O(CH₂)₄O(C₃H₆O)₄(C₂H₄O)₂₀H;

Emulsogen® R307 which is a random ethoxylated/propoxylated 1,4-butanediol vinyl ether having the empirical formula:

CH₂═CH—O(CH₂)₄O(C₃H₆O)₄(C₂H₄O)₃₀H;

Emulsogen® RAL 109 which is a random ethoxylated/propoxylated allyl ether having the empirical formula:

CH₂═CHCH₂—O(C₃H₆O)₄(C₂H₄O)₁₀H;

Emulsogen® RAL 208 which is a random ethoxylated/propoxylated allyl ether having the empirical formula:

CH₂═CHCH₂—O(C₃H₆O)₄(C₂H₄O)₂₀H;

Emulsogen® RAL 307 which is a random ethoxylated/propoxylated allyl ether having the empirical formula:

CH₂═CHCH₂═CHCH₂—O(C₃H₆O)₄(C₂H₄O)₃₀H;

Maxemul® 5010 which is a hydrophobic carboxylated C₁₂-C₁₅ alkenyl ethoxylated with 24 ethylene oxide units;
Maxemul® 5011 which is a hydrophobic carboxylated C₁₂-C₁₅ alkenyl ethoxylated with 34 ethylene oxide units;
and BX-AA-E5P5 which is a random ethoxylated/propoxylated allyl ether having the empirical formula:

CH₂═CHCH₂—O(C₃H₆O)₅(C₂H₄O)₅H.

The amount of the at least one semi-hydrophobic surfactant vinyl monomer used in the preparation of the at least one cationic polymer (i) used in the composition according to the present disclosure may vary to a large extent and depends, among other things, on the final rheological properties desired for the polymer.

When present, the at least one semi-hydrophobic surfactant vinyl monomer is present in an amount ranging from 0.01% to 25% by weight, such as from 0.1% to 10% by weight, relative to the total weight of the mixture of monomers.

The at least one cationic polymer (i) used in the composition according to the present disclosure is prepared from a mixture of monomers that may comprise at least one hydroxylated nonionic vinyl monomers.

These monomers are monomers comprising an ethylenic unsaturation or unsaturations and comprising at least one hydroxyl substituent.

Non-limiting examples of hydroxylated nonionic vinyl monomers include hydroxylated C₁-C₆ alkyl (meth)acrylates, such as hydroxylated C₁-C₄ alkyl (meth)acrylates, for example 2-hydroxyethyl methacrylate (HE MA), 2-hydroxyethyl acrylate (2-HEA), or 3-hydroxypropyl acrylate; and hydroxylated C₁-C₄ alkyl (meth)acrylamides, such as N-(2-hydroxyethyl)methacrylamide, N-(2-hydroxyethyl)acrylamide, N-(3-hydroxypropyl)acrylamide, or N-(2,3-dihydroxypropyl)acrylamide. In at least one embodiment the at least one hydroxylated nonionic vinyl monomer is chosen from allyl alcohol, glycerol monoallyl ether, 3-methyl-3-buten-1-ol, vinyl alcohol precursors and equivalents thereof, such as vinyl acetate.

When it is present, the at least one hydroxylated nonionic vinyl monomer may be present in an amount of up to 10% by weight of the total weight of the mixture of monomers. In at least one embodiment, the at least one hydroxylated nonionic vinyl monomer is present in an amount ranging from 0.01% to 10% by weight, such as from 1% to 8%, and such as from 1% to 5% by weight, relative to the total weight of the mixture of monomers.

The at least one cationic polymer (i) used in the composition according to the present disclosure is prepared from a mixture of monomers which may comprise at least one crosslinking monomer for introducing branches or for controlling the molecular mass.

Polyunsaturated crosslinking agents that can be used are well known in the prior art. Monounsaturated compounds having a reactive group capable of crosslinking a copolymer formed, before, during, or after the polymerization can also be used. Other crosslinking monomers that can be used may be polyfunctional monomers containing multiple reactive groups, such as epoxide or isocyanate groups and hydrolysable silane groups. Numerous polyunsaturated compounds may be used to generate a partially or substantially crosslinked three-dimensional network.

Non-limiting examples of polyunsaturated crosslinking monomers that can be used are polyunsaturated aromatic monomers, such as divinylbenzene, divinyl naphthylene and trivinylbenzene; polyunsaturated alicyclic monomers, such as 1,2,4-trivinylcyclohexane; difunctional phthalic acid esters, such as diallyl phthalate; polyunsaturated aliphatic monomers, such as dienes, trienes, and tetraenes, including isoprene, butadiene, 1,5-hexadiene, 1,5,9-decatriene, 1,9-decadiene, and 1,5-heptadiene.

Other exemplary polyunsaturated crosslinking monomers that can be used are polyalkenyl ethers, such as triallyl pentaerythritol, diallyl pentaerythritol, diallyl sucrose, octaallyi sucrose, and trimethylolpropane diallyl ether; polyunsaturated esters of polyalcohols or of polyacids, such as 1,6-hexanediol di(meth)acrylate, tetramethylene tri(meth)acrylate, allyl acrylate, diallyl itaconate, diallyl fumarate, diallyl maleate, trimethylolpropane tri(meth)acrylate, trimethylolpropane di(meth)acrylate, and polyethylene glycol di(meth)acrylate; alkylenebisacrylamides, such as methylenebisacrylamide or propylenebisacrylamide; hydroxylated and carboxylated derivatives of methylenebisacrylamide, such as N,N′-bismethylolmethylenebisacrylamide; polyethylene glycol di(meth)acrylates, such as ethylene glycol di(meth)acrylate, diethylene glycol di(meth)acrylate, triethylene glycol di(meth)acrylate, polyunsaturated silanes such as dimethyldivinylsilane, methyltrivinylsilane, allyldimethylvinylsilane, diallyldimethylsilane, and tetravinylsilane, and polyunsaturated stannanes, such as tetraallyl tin and diallyldimethyl tin.

Exemplary monounsaturated crosslinking monomers that can be used and that bear a reactive group may be N-methylolacrylamides; N-alkoxy(meth)acrylamides, where the alkoxy group is a C₁-C₁₈ group; and unsaturated hydrolysable silanes such as triethoxyvinylsilane, trisisopropoxyvinylsilane, and 3-triethoxysilyipropyl methacrylate.

Exemplary polyfunctional crosslinking monomers that can be used and that contain several reactive groups may be hydrolysable silanes such as ethyltriethoxysilane and ethyltrimethoxysilane; hydrolysable epoxy silanes, such as 2-(3,4-epoxycyclohexyl)ethyl triethoxysilane and 3-glycidoxypropyltrimethoxysilane; polyisocyanates, such as 1,4-diisocyanatobutane, 1,6-diisocyanatohexane, 1,4-phenylene diisocyanate, and 4,4′-oxybis(phenyl isocyanate); unsaturated epoxides, such as glycidyl methacrylate and allylglycidyl ether; polyepoxides, such as diglycidyl ether, 1,2,5,6-diepoxyhexane, and ethylene glycol diglycidyl ether.

Exemplary polyunsaturated crosslinking monomers that can be used are ethoxylated polyols, such as diols, triols, and bisphenols, ethoxylated with 2 to 100 mol of ethylene oxide per mole of hydroxyl functional group and ending with a polymerizable unsaturated group, such as a vinyl ether, an allyl ether, an acrylate ester, or a methacrylate ester. Such crosslinking monomers may, for example, be ethoxylated bisphenol A dimethacrylate, ethoxylated bisphenol F dimethacrylate, and ethoxylated trimethylolpropane trimethacrylate.

Other exemplary ethoxylated crosslinking monomers that can be used in the present disclosure are the ethoxylated polyol-derived crosslinking agents disclosed in U.S. Pat. No. 6,140,435.

In one aspect of the disclosure, crosslinking monomers include polyol acrylate and methacrylate esters having at least two acrylate or methacrylate ester groups, such as trimethylolpropane triacrylate (TMPTA), trimethylolpropane dimethacrylate, triethylene glycol dimethacrylate (TEGDMA), and ethoxylated (30) bisphenol A dimethacrylate (EOBDMA).

When it is present, the at least one crosslinking monomer is present in an amount of at most 5% by weight relative to the weight of the mixture of monomers. According to at least one embodiment, the crosslinking monomers are present in an amount ranging from 0.001% to 5% by weight, such as from 0.05% to 2% by weight, such as from 0.1% to 1% by weight, relative to the total weight of the mixture of monomers.

The mixture of monomers may comprise at least one chain-transfer agent. The at least one chain-transfer agent is a compound well known in the prior art.

Non-limiting examples include thiolated compounds, disulfide compounds, such as C₁-C₁₈ mercaptans, mercaptocarboxylic acids, mercaptocarboxylic acid esters, thioesters, C₁-C₁₈ alkyl disulfides, aryl disulfides, polyfunctional thiols; phosphites and hypophosphites; haloalkyl compounds, such as carbon tetrachloride, bromotrichloromethane; and unsaturated chain-transfer agents, such as alpha-methylstyrene.

The polyfunctional thiols are, for example, trifunctional thiols, such as trimethylolpropane tris(3-mercaptopropionate), tetrafunctional thiols, such as pentaerythritol tetra(3-mercaptopropionate), pentaerythritol tetra(thioglycolate), and pentaeryth ritol tetra(thiolactate); and hexafunctional thiols, such as pentaerythritol hexa(thioglyconate).

In at least one embodiment, the at least one chain-transfer agent may be a catalytic chain-transfer agent which reduces the molecular weight of the addition polymers during the free-radical polymerization of the vinyl monomers. Non-limiting examples include cobalt complexes, such as cobalt (II) chelates. The at least one catalytic chain-transfer agent can often be used at low concentrations relative to the thiolated chain-transfer agents.

In at least one embodiment, the chain-transfer agent may be made of octyl mercaptan, n-dodecyl mercaptan, t-dodecyl mercaptan, hexadecyl mercaptan, octadecyl mercaptan (ODM), isooctyl 3-mercaptopropionate (IMP), butyl 3-mercaptopropionate, 3-mercaptopropionic acid, butyl thioglycolate, isooctyl thioglycolate, and dodecyl thioglycolate.

When it is present, the at least one chain-transfer agent is added to the mixture of monomers in an amount up to 10% by weight relative to the total weight of the mixture of monomers. In at least one embodiment, the at least one chain-transfer agent is present in an amount ranging from 0.1% to 5% by weight, relative to the total weight of monomers.

The mixture of monomers for preparing the at least one cationic polymer (i) used in the composition according to the present disclosure may comprise at least one polymeric stabilizer for obtaining stable dispersions or emulsions. In at least one embodiment, the polymers are water-soluble. Non-limiting examples include synthetic polymers, such as polyvinyl alcohols, partially hydrolysed polyvinyl acetates, polyvinylpyrrolidone, polyacrylamides, polymethacrylamides, carboxylated addition polymers, poly(alkyl vinyl ether)s; water-soluble natural polymers, such as gelatine, peptins, alginates, casein; and modified natural polymers, such as methylcellulose, hydroxypropylcellulose, carboxymethylcellulose, and allyl hydroxyethylcelluloses.

The at least one polymeric stabilizer is used in an amount at most equal to 2% by weight relative to the total weight of the emulsion, such as an amount ranging from 0.0001% to 1% by weight, and such as from 0.01% to 0.5% by weight, relative to the total weight of the mixture of monomers.

According to at least one embodiment, the mixture of monomers comprises, relative to the total weight of the mixture of monomers:
a) from 10% to 70% by weight of the at least one vinyl monomer substituted with at least one amino group,
b) from 20% to 80% by weight of the at least one hydrophobic nonionic vinyl monomer,
c) from 0.001% to 25% by weight of the at least one associative vinyl monomer,
d) from 0 to 25% by weight of the at least one semi-hydrophobic surfactant vinyl monomer,
e) from 0 to 10% by weight of at least one hydroxylated non ionic vinyl monomer,
f) from 0 to 5% by weight of at least one crosslinking monomer,
g) from 0 to 10% by weight of at least one chain-transfer agent, and
h) from 0 to 2% by weight of at least one polymeric stabilizer.

In another embodiment, the mixture of monomers comprises, relative to the total weight of the mixture of monomers:
a) from 20% to 60% by weight of the at least one vinyl monomer substituted with at least one amino group,
b) from 20% to 70% by weight of the at least one hydrophobic nonionic vinyl monomer,
c) from 0.01% to 15% by weight of the at least one associative vinyl monomer,
d) from 0.1% to 10% by weight of the at least one semi-hydrophobic surfactant vinyl monomer,
e) from 0.01% to 10% by weight of the at least one hydroxylated non ionic vinyl monomer,
f) from 0.001% to 5% by weight of the at least one crosslinking monomer,
g) from 0.001% to 10% by weight of the at least one chain-transfer agent, and
h) from 0 to 2% by weight of the at least one polymeric stabilizer.

In another embodiment, the mixture of monomers for preparing the at least one cationic polymer (i) used in the composition according to the present disclosure comprises, relative to the total weight of the mixture of monomers:
a) from 20% to 50% by weight of at least one vinyl monomer substituted with at least one amino group chosen from:
3-(N,N-dimethylamino)propyl (meth)acrylate,
N′-(3-N,N-dimethylamino)propyl (meth)acrylamide,
2-(N,N-dimethylamino)ethyl (meth)acrylate,
2-(N,N-diethylamino)ethyl (meth)acrylate,
2-(tert-butylamino)ethyl (meth)acrylate,
2-(N,N-dimethylamino)propyl(meth)acrylamide, and
2-(N,N-dimethylamino)neopentyl acrylate,
   b) from 50% to 65% by weight of at least one hydrophobic nonionic vinyl monomer chosen from acrylic acid C₁-C₃₀ alkyl esters and methacrylic acid C₁-C₃₀ alkyl esters,
   c) from 0.1% to 10% by weight of at least one associative vinyl monomer chosen from polyethoxylated cetyl (meth)acrylates, polyethoxylated cetearyl methacrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-(1′-phenylethyl)phenyl (meth)acrylates, polyethoxylated (meth)acrylates of hydrogenated castor oil, polyethoxylated canola (meth)acrylates, and polyethoxylated (meth)acrylates of cholesterol,
   d) from 0.1% to 10% by weight of at least one semi-hydrophobic surfactant vinyl monomer having one of the following formulae:

CH₂═CH—O(CH₂)ₐO(C₃H₆O)_{b}(C₂H₄O)_{c}H or

CH₂═CHCH₂O(C₃H₆O)_{d}(C₂H₄O)ₑH;

wherein:

a is an integer ranging from 2 to 4;

b is an integer ranging from 1 to 10;

c is an integer ranging from 5 to 50;

d is an integer ranging from 1 to 10; and

e is an integer ranging from 5 to 50,

e) up to 10% by weight of at least one hydroxylated nonionic vinyl monomer,

f) up to 5% by weight of at least one crosslinking monomer,

g) up to 10% by weight of at least one chain-transfer agent, and

h) up to 2% by weight of at least one polymeric stabilizer.

Non-limiting exemplary cationic polymers (i) according to the present disclosure are polymers derived from the polymerization of the mixture of monomers below:

a di(C₁-C₄ alkyl)amino(C₁-C₆ alkyl)methacrylate,

at least one (meth)acrylic acid C₁-C₃₀ alkyl ester,

a polyethoxylated C₁₀-C₃₀ alkyl methacrylate with 20 to 30 mol of ethylene oxide,

a polyethylene glycol/polypropylene glycol 30/5 alkyl ether,

a hydroxy(C₂-C₆ alkyl)methacrylate,

an ethylene glycol dimethacrylate.

Among the at least one cationic polymers (i) used in the composition according to the present disclosure, non-limiting mention may be made of the compound sold by the company Noveon under the name Qarbopol Aqua CC Polymer and which corresponds to the INCI name Polyacrylate-1 Crosspolymer.

The Polyacrylate-1 Crosspolymer is the product of the polymerization of a mixture of monomers comprising (or constituted of):

a di(C₁-C₄ alkyl)amino(C₁-C₆ alkyl)methacrylate,

at least one (meth)acrylic acid C₁-C₃₀ alkyl ester,

a polyethoxylated C₁₀-C₃₀ alkyl methacrylate (20-25 mol of ethylene oxide unit),

a polyethylene glycol/polypropylene glycol 30/5 alkyl ether,

a hydroxyl(C₂-C₆ alkyl)methacrylate, and

an ethylene glycol dimethacrylate.

The at least one cationic polymer (i) used in the compositions according to the present disclosure is present in an amount ranging from 0.01% to 10% by weight, such as from 0.05% to 5% by weight, and such as from 0.1% to 1% by weight, relative to the total weight of the composition.

The at least one cationic polymer (i) used in the composition according to the present disclosure may be prepared by conventional polymerization techniques, such as emulsion polymerization, as is well known in the polymer field. The polymerization may be carried out by a simple batch process or by a controlled addition process, or else the reaction can be initiated in a small reactor and then the bulk of the monomers can be added to the reactor in a controlled manner (seeding process). In at least one embodiment, the polymerization is carried out at a reaction temperature ranging from 20° C. to 80° C., even though higher or lower temperatures may be used. To facilitate the emulsification of the mixture of monomers, the emulsion polymerization is carried out in the presence of at least one surfactant, present in an amount ranging from 1% to 10% by weight, such as from 3% to 8% by weight, and such as from 5% to 7% by weight, relative to the total weight of the emulsion. The emulsion polymerization reaction medium also comprises at least one radical initiator, which may be in an amount ranging from 0.01% to 3% by weight, relative to the total weight of the mixture of monomers. The polymerization can be carried out in an aqueous medium or an aqueous-alcoholic medium at a neutral or weakly alkaline pH.

In an exemplary polymerization, the mixture of monomers is added with stirring to a solution of emulsifying surfactants, such as a nonionic surfactant, for example a linear or branched alcohol ethoxylate, or a mixture of nonionic and anionic surfactants, such as fatty alcohol sulfates or fatty alcohol alkyl sulfonates, in a suitable amount of water, in a suitable reactor, for preparing the monomer emulsion. The emulsion is deoxygenated by means of any known method, and the polymerization reaction is then initiated by adding a polymerization catalyst (initiator), such as sodium persulfate, or any other suitable addition polymerization catalyst, as is well known in the polymer field. The reaction is stirred until the polymerization is complete, generally for a period ranging from 4 hours to 16 hours. The monomer emulsion can be heated to a temperature ranging from 20° C. to 80° C. before the addition of the initiator, if so desired. The amount of monomers having not reacted can be eliminated by the addition of an additional amount of catalyst. The polymer emulsion obtained can be withdrawn from the reactor and packaged so as to be stored or used. In at least one embodiment, the pH or other physical or chemical characteristics of the emulsion can be adjusted before the emulsion is withdrawn from the reactor. The emulsion produced has a total solids content which ranges from 10% to 40% by weight. The total amount of polymers in the emulsion obtained ranges from 15% to 35% by weight, and is in at least one embodiment at most 25% by weight.

Surfactants suitable for facilitating the emulsion polymerization may be chosen from nonionic, anionic, amphoteric, and cationic surfactants. In at least one embodiment, the at least one surfactant is chosen from nonionic and anionic surfactants.

All types of nonionic, anionic, amphoteric, and cationic surfactants conventionally used in emulsion polymerizations may be used.

The polymerization may be carried out in the presence of at least one free-radical initiator. The latter may be chosen from insoluble inorganic persulfate compounds, such as ammonium persulfate, potassium persulfate, and sodium persulfate; peroxides such as hydrogen peroxide, benzoyl peroxide, acetyl peroxide, and lauryl peroxide; organic hydroperoxides, such as cumen hydroperoxide and t-butyl hydroperoxide; organic peracids, such as peracetic acid; and oil-soluble free-radical-producing agents, such as 2,2′-azobisisobutyronitrile. The peroxides and the peracids may be optionally activated with reducing agents, such as sodium bisulfite or ascorbic acid, transition metals, or hydrazine. Free-radical initiators which are suitable include, but are not limited to, water-soluble azo polymerization initiators, such as 2,2′-azobis(tert-alkyl) compounds having a water-soluble substituent on the alkyl group. Exemplary azo polymerization catalysts include the free-radical initiators Vazo®, sold by the company DuPont, such as Vazo® 44 (2,2′-azobis(2,4,5-dihydroimidazolyl)propane), Vazo® 56 (2,2′-azobis(2-methylpropionamidine) dihydro-chloride) and Vazo® 68 (4,4′-azobis(4-cyanovaleric acid)).

Non-limiting examples of the at least one associative polymers of anionic type that may be used according to the present disclosure include:

(I) copolymers comprising at least one hydrophilic unit and at least one fatty-chain allylic ether unit, such as those whose hydrophilic unit is chosen from an ethylenic unsaturated anionic monomer, for example, a vinylcarboxylic acid, an acrylic acid, and a methacrylic acid, the fatty-chain allylic ether unit of which corresponds to the monomer of formula (VI) below:

CH₂═C(R′)CH₂—O—Bₙ—R (VI)

wherein R′ is a hydrogen atom or CH₃, B is an ethyleneoxy radical, n is zero or is an integer ranging from 1 to 100, R is a hydrocarbon-based radical chosen from alkyl, arylalkyl, aryl, alkylaryl, and cycloalkyl radicals, containing from 8 to 30 carbon atoms, such as from 10 to 24 carbon atoms, such as from 12 to 18 carbon atoms. In at least one embodiment, a unit of formula (VI) is a unit wherein R′ is a hydrogen atom, n is equal to 10, and R is a stearyl (C₁₈) radical.

Non-limiting examples include copolymers of methacrylic acid and of allylic ethers of C₈-C₃₀ fatty alcohols.

Anionic associative polymers of this type are described and prepared, according to an emulsion polymerization process, in patent EP0 216 479.

In at least one embodiment, the at least one anionic associative polymer according to the present disclosure is chosen from polymers formed from 20% to 60% by weight of acrylic acid and/or of methacrylic acid, from 5% to 60% by weight of lower alkyl (meth)acrylates, from 2% to 50% by weight of fatty-chain allylic ether of formula (I), and from 0% to 1% by weight of a crosslinking agent which is a well-known copolymerizable unsaturated polyethylenic monomer, for instance diallyl phthalate, allyl (meth)acrylate, divinylbenzene, (poly)ethylene glycol dimethacrylate, or methylenebisacrylamide.

Exemplary polymers include, but are not limited to, crosslinked terpolymers of methacrylic acid, of ethyl acrylate, and of polyethylene glycol (10 EO) stearyl alcohol ether (Steareth-10), such as those sold by the company Ciba under the names Salcare SC 80® and Salcare SC 90®, which are aqueous 30% emulsions of a crosslinked terpolymer of methacrylic acid, of ethyl acrylate, and of steareth-10 allylic ether (40/50/10).

(II) copolymers comprising at least one hydrophilic unit of unsaturated olefinic carboxylic acid type, and at least one hydrophobic unit of (C₁₀-C₃₀)alkyl ester of unsaturated carboxylic acid type.

These Polymers are Chosen from Those Wherein the Hydrophilic Unit of unsaturated olefinic carboxylic acid type corresponds to the monomer of formula (VII) below:

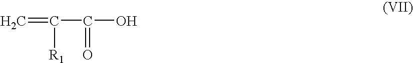

wherein R₁ is a hydrogen atom, CH₃, or C₂H₅, that is to say acrylic acid, methacrylic acid, or ethacrylic acid units, and wherein the hydrophobic unit of (C₁₀-C₃₀)alkyl ester of unsaturated carboxylic acid type corresponds to the monomer of formula (III) below:

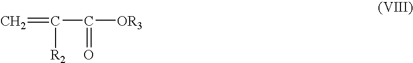

wherein R₂ is a hydrogen atom, CH₃, or C₂H₅, that is to say acrylate, methacrylate, or ethacrylate units, R₃ is a C₁₀-C₃₀ radical, such as a C₁₂-C₂₂ alkyl radical.

Exemplary copolymers include, but are not limited to, copolymers of unsaturated carboxylic acids and of unsaturated C₁₀-C₃₀ carboxylates.

(C₁₀-C₃₀) alkyl esters of unsaturated carboxylic acids according to the present disclosure include, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate, and dodecyl methacrylate.

Anionic polymers of this type are described and prepared, for example, according to U.S. Pat. Nos. 3,915,921 and 4,509,949.

In at least one embodiment, the at least one anionic associative polymer of this type that will be used includes polymers formed from a monomer mixture comprising:

(i) acrylic acid,

(ii) an ester of formula (VIII) described above wherein R₂ is a hydrogen atom or CH₃, R₃ is an alkyl radical containing from 12 to 22 carbon atoms,

(iii) and a crosslinking agent, which is a well-known copolymerizable polyethylenic unsaturated monomer, for instance diallyl phthalate, allyl (meth)acrylate, divinylbenzene, (poly)ethylene glycol dimethacrylate, and methylenebisacrylamide.

Non-limiting examples of the at least one anionic associative polymer of this type include those comprising from 95% to 60% by weight of acrylic acid (hydrophilic unit), 4% to 40% by weight of G₁₀-C₃₀ alkyl acrylate (hydrophobic unit), and 0% to 6% by weight of crosslinking polymerizable monomer, or those consisting of from 98% to 96% by weight of acrylic acid (hydrophilic unit), 1% to 4% by weight of C₁₀-C₃₀ alkyl acrylate (hydrophobic unit), and 0.1% to 0.6% by weight of crosslinking polymerizable monomer, such as those described above.

Non-limiting examples of the said above polymers include the products sold by the company Goodrich under the trade names Pemulen TR1®, Pemulen TR2®, and Carbopol 1382®, and the product sold by the company SEPPIC under the name Coatex sx.

(III) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608® by the company Newphase Technologies.

(IV) acrylic terpolymers obtained from (a) an α,β-ethylenically unsaturated carboxylic acid, (b) a non-surfactant α,β-ethylenically unsaturated monomer other than (a), and (c) a nonionic surfactant monomer obtained by reacting an ethylenically unsaturated monoisocyanate with a monohydric surfactant.

For example, such polymers may be obtained from an α,β-ethylenically unsaturated carboxylic acid, and from a nonionic urethane monomer that is the product of reaction of a monohydric nonionic amphiphilic compound with a monoethylenically unsaturated isocyanate.

In at least one embodiment, such a polymer may comprise, relative to the total weight of the terpolymer:

(a) from about 20% to 70% by weight, such as from 25% to 55% by weight of an α,β-ethylenically unsaturated carboxylic acid,

(b) from about 20% to 80% by weight, such as from 30% to 65% by weight of a non-surfactant ethylenically unsaturated monomer other than (a), and

(c) from about 0.5% to 60% by weight, such as from 10% to 50% by weight of a nonionic urethane monomer that is the product of reaction of a monohydric nonionic amphiphilic compound with a monoethylenically unsaturated isocyanate.

An exemplary polymer includes Viscophobe DB 1000, sold by Amerchol.

(V) copolymers comprising among their monomers a carboxylic acid containing α,β-monoethylenic unsaturation and an ester of a carboxylic acid containing α,β-monoethylenic unsaturation and of an oxyalkylenated fatty alcohol.

These compounds may also comprise as monomers an ester of a carboxylic acid containing α,β-monoethylenic unsaturation and of a C₁-C₄ alcohol.

Examples of compounds of this type include Aculyn 22® sold by the company Rohm & Haas, which is a methacrylic acid/ethyl acrylate/stearyl methacrylate oxyalkylenated terpolymer, and Aculyn 28 (terpolymer of methacrylic acid/ethyl acrylate/oxyethylenated (25 EO) behenyl methacrylate).

(VI) amphiphilic polymers comprising at least one ethylenically unsaturated monomer containing a sulfonic group, the polymer comprising at least one hydrophobic part.

The expression “amphiphilic polymer” means any polymer comprising both a hydrophilic part and a hydrophobic part, such as a fatty chain, for example, a fatty chain containing from 8 to 30 carbon atoms.

The hydrophobic part present in the polymers of the present disclosure contains from 6 to 50 carbon atoms, such as from 6 to 22 carbon atoms, such as from 6 to 18 carbon atoms, and such as from 12 to 18 carbon atoms.

The polymers in accordance with the present disclosure, in at least one embodiment, are partially or totally neutralized with a mineral base (sodium hydroxide, potassium hydroxide or aqueous ammonia) or an organic base such as mono-, di-, or triethanolamine, an aminomethylpropanediol, N-methylglucamine, basic amino acids, for instance arginine and lysine, and mixtures of these compounds.

The amphiphilic polymers in accordance with the present disclosure have a number-average molecular weight ranging from 1000 g/mol to 20 000 000 g/mol, such as from 20 000 g/mol to 5 000 000 g/mol, and such as from 100 000 g/mol to 1 500 000 g/mol.

The amphiphilic polymers according to the present disclosure may or may not be crosslinked.

In at least one embodiment, crosslinked amphiphilic polymers are chosen.

When they are crosslinked, the crosslinking agents may be chosen from compounds containing olefinic polyunsaturation commonly used for the crosslinking of polymers obtained by radical polymerization.

Non-limiting examples of crosslinking agents include divinylbenzene, diallyl ether, dipropylene glycol diallyl ether, polyglycol diallyl ethers, triethylene glycol divinyl ether, hydroquinone diallyl ether, ethylene glycol di(meth)acrylate or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allyl or vinyl ethers of polyfunctional alcohols, and also allylic esters of phosphoric and/or vinylphosphonic acid derivatives, or mixtures of these compounds.

In at least one embodiment, methylenebisacrylamide, allyl methacrylate, or trimethylolpropane triacrylate (TMPTA) will be used. The degree of crosslinking will range from 0.01 mol % to 10 mol %, such as from 0.2 mol % to 2 mol %, relative to the polymer. The ethylenically unsaturated monomers containing a sulfonic group may be chosen from vinylsulfonic acid, styrenesulfonic acid, (meth)acrylamido(C₁-C₂₂)alkylsulfonic acids, N—(C₁-C₂₂)alkyl(meth)acrylamido(C₁-C₂₂)alkylsulfonic acids, for instance undecylacrylamidomethanesulfonic acid, and also partially or totally neutralized forms thereof.

In at least one embodiment, the at least one ethylenically unsaturated monomer containing a sulfonic group will be chosen from (Meth)acrylamido(C₁-C₂₂)alkylsulfonic acids such as, for example, acrylamidomethanesulfonic acid, acrylamido-ethanesulfonic acid, acrylamidopropanesulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid or 2-acrylamido-2,6-dimethyl-3-heptanesulfonic acid, and also partially or totally neutralized forms thereof.

In at least one embodiment, 2-Acrylamido-2-methylpropanesulfonic acid (AMPS), and/or its partially or totally neutralized forms will be used. The at least one amphiphilic polymer in accordance with the present disclosure may be chosen from random amphiphilic polymers of AMPS modified by reaction with a C₆-C₂₂ n-monoalkylamine or di-n-alkylamine, such as those described in patent application WO 00/31154. These polymers may also contain other ethylenically unsaturated hydrophilic monomers chosen, for example, from (meth)acrylic acids, β-substituted alkyl derivatives thereof, and esters thereof obtained with monoalcohols or mono- or polyalkylene glycols, (meth)acrylamides, vinylpyrrolidone, maleic anhydride, itaconic acid, and maleic acid.

Non-limiting examples of the polymers of the present disclosure are chosen from amphiphilic copolymers of AMPS and of at least one ethylenically unsaturated hydrophobic monomer comprising at least one hydrophobic portion containing from 6 to 50 carbon atoms, such as from 6 to 22 carbon atoms, such as from 6 to 18 carbon atoms, and such as 12 to 18 carbon atoms.

These same copolymers may also contain at least one ethylenically unsaturated monomer not comprising a fatty chain chosen from (meth)acrylic acids, β-substituted alkyl derivatives thereof, esters thereof obtained with monoalcohols or mono- or polyalkylene glycols, (meth)acrylamides, vinylpyrrolidone, maleic anhydride, itaconic acid, and maleic acid.

These copolymers are described, for example, in patent application EP-A-750 899, U.S. Pat. No. 5,089,578, and in the following publications from Yotaro Morishima:

“Self-assembling amphiphilic polyelectrolytes and their nanostructures” Chinese Journal of Polymer Science (2000) 18(40): 323-336;

“Micelle formation of random copolymers of sodium 2-(acrylamido)-2-methylpropanesulfonate and a non-ionic surfactant macromonomer in water as studied by fluorescence and dynamic light scattering” Macromolecules (2000) 33(10):3694-3704;

“Solution properties of micelle networks formed by non-ionic moieties covalently bound to a polyelectrolyte: salt effects on rheological behavior” Langmuir (2000) 16(12):5324-5332; and

“Stimuli responsive amphiphilic copolymers of sodium 2-(acrylamido)-2-methylpropanesulfonate and associative macromonomers” Potym. Preprint, Div. Polym. Chem. (1999) 40(2):220-221.

In at least one embodiment, the at least one ethylenically unsaturated hydrophobic monomer of these particular copolymers is chosen from the acrylates and acrylamides of formula (IX) below:

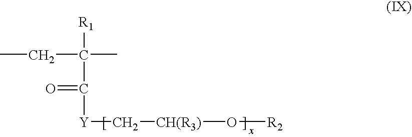

wherein R₁ and R₃, which may be identical or different, are a hydrogen atom or a linear or branched C₁-C₆ alkyl radical, such as methyl; Y is O or NH; R₂ is a hydrophobic hydrocarbon-based radical containing from 6 to 50 carbon atoms, such as from 6 to 22 carbon atoms, such as from 6 to 18 carbon atoms, and such as from 12 to 18 carbon atoms; x is a number of moles of alkylene oxide ranging from 0 to 100.

The radical R₂ is chosen from linear C₆-C₁₈ alkyl radicals (for example n-hexyl, n-octyl, n-decyl, n-hexadecyl and n-dodecyl) and branched or cyclic C₆-C₁₈ alkyl radicals (for example cyclododecane (C₁₂) or adamantane (C₁₀)); C₆-C₁₈ alkylperfluoro radicals (for example the group of formula —(CH₂)₂—(CF₂)₉—CF₃); the cholesteryl radical (C₂₇) or a cholesterol ester residue, for instance the cholesteryl oxyhexanoate group; and aromatic polycyclic groups, for instance naphthalene or pyrene. In at least one embodiment, the radical is chosen from linear alkyl radicals, such as the n-dodecyl radical.

According to one embodiment of the present disclosure, the monomer of formula (IX) comprises at least one alkylene oxide unit (x≧1) and a polyoxyalkylenated chain. The polyoxyalkylenated chain is constituted of ethylene oxide units and/or propylene oxide units. The number of oxyalkylenated units ranges from 3 to 100, such as from 3 to 50, and such as from 7 to 25.

Non-limiting examples of these polymers include

crosslinked or non-crosslinked, neutralized or non-neutralized copolymers comprising from 15% to 60% by weight of AMPS units and from 40% to 85% by weight of (C₈-C₁₆)alkyl(meth)acrylamide units or of (C₈-C₁₆)alkyl (meth)acrylate units relative to the polymer, such as those described in patent application EP-A-750 899; and

terpolymers comprising from 10 mol % to 90 mol % of acrylamide units, from 0.1 mol % to 10 mol % of AMPS units, and from 5 mol % to 80 mol % of n-(C₆-C₁₈)alkylacrylamide units, such as those described in U.S. Pat. No. 5,089,578.

Non-limiting mention may also be made of copolymers of totally neutralized AMPS and of dodecyl methacrylate, and also crosslinked and non-crosslinked copolymers of AMPS and of n-dodecylmethacrylamide, such as those described in the Morishima articles mentioned above.

In an exemplary embodiment, the copolymers constitute 2-acrylamido-2-methylpropanesulfonic acid (AMPS) units of formula (X) below:

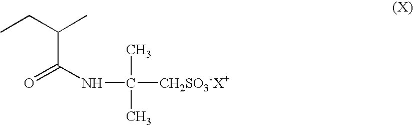

wherein X⁺ is chosen from a proton, an alkali metal cation, an alkaline-earth metal cation, and the ammonium ion,
and of units of formula (XI) below:

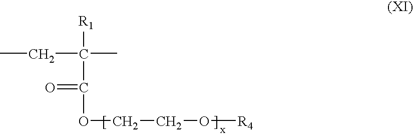

wherein x is an integer ranging from 3 to 100, such as from 5 to 80, and such as from 7 to 25; R₁ has the same meaning as that given above in formula (IX), and R₄ is a linear or branched C₆-C₂₂, such as a C₁₀-C₂₂ alkyl.

In at least one embodiment, the polymers are those for which x=25, R₁ is methyl, and R₄ is n-dodecyl, such as those described in the Morishima articles mentioned above.

In at least one embodiment, X⁺ is sodium or ammonium.

The amphiphilic polymers in accordance with the present disclosure may be obtained according to the standard radical polymerization processes in the presence of at least one initiator such as, for example, azobisisobutyronitrile (AIBN), azobisdimethylvalero-nitrile, 2,2-azobis[2-amidinopropane]hydrochloride ABAH, organic peroxides such as dilauryl peroxide, benzoyl peroxide, tert-butyl hydroperoxide, etc., mineral peroxide compounds such as potassium persulfate or ammonium persulfate, or H₂O₂ optionally in the presence of reducing agents.

The said amphiphilic polymers are obtained, for example, by radical polymerization in tert-butanol medium wherein they precipitate.

By using precipitation polymerization in tert-butanol, it is possible to obtain a particle size distribution of the polymer that is desired for its uses.

The size distribution of the polymer particles may be determined, for example, by laser diffraction or image analysis.

In at least one embodiment, the distribution for this type of polymer, determined by image analysis, is as follows: 60.2% less than 423 microns, 52.0% less than 212 microns, 26.6% less than 106 microns, 2.6% less than 45 microns, and 26.6% greater than 850 microns.

The reaction may be performed at a temperature ranging from 0° C. to 150° C., such as from 10° C. to 100° C., either at atmospheric pressure or under reduced pressure. It may also be performed under inert atmosphere, such as under nitrogen.

According to this process, 2-acrylamido-2-methylpropanesulfonic acid (AMPS) or a sodium or ammonium salt thereof was especially polymerized with a (meth)acrylic acid ester and

a C₁₀-C₁₈ alcohol oxyethylenated with 8 mol of ethylene oxide (Genapol® C-080 from the company Hoechst/Clariant),

a C₁₁ oxo alcohol oxyethylenated with 8 mol of ethylene oxide (Genapol® UD-080 from the company Hoechst/Clariant),

a C₁₁ oxo alcohol oxyethylenated with 7 mol of ethylene oxide (Genapol®G UD-070 from the company Hoechst/Clariant),

a C₁₂-C₁₄ alcohol oxyethylenated with 7 mol of ethylene oxide (Genapol® LA-070 from the company Hoechst/Clariant), or

a C₁₂-C₁₄ alcohol oxyethylenated with 9 mol of ethylene oxide (Genapol® LA-090 from the company Hoechst/Clariant).

The monomer distribution in the polymers of the present disclosure may be, for example, alternating, block (including multiblock), or random.

According to the present disclosure, the polymers may contain heat-sensitive pendent chains and for the aqueous solution thereof to have a viscosity that, beyond a certain threshold temperature, increases or remains virtually constant as the temperature increases.

Polymers of the present disclosure include those whose aqueous solution has a viscosity that is low below a first threshold temperature and that, above this first threshold temperature, increases to a maximum as the temperature increases, and that, above a second threshold temperature, decreases again as the temperature increases. For example, the viscosity of the polymer solutions below the first threshold temperature may be from 5% to 50%, such as from 10% to 30%, of the maximum viscosity at the second threshold temperature.

These polymers may lead, in water, to a phenomenon of demixing by heating, reflected by curves showing, as a function of the temperature and the concentration, a minimum known as the LCST (Lower Critical Solution Temperature).

The viscosities (measured at 25° C. using a Brookfield viscometer, needle No. 7) of the aqueous 1% solutions range from 20 000 m Pa·s to 100 000 mPa·s, such as from 60 000 mPa·s to 70 000 mPa·s.

According to the present disclosure, the at least one anionic associative polymer may be present in an amount ranging from 0.01% to 20% by weight, such as from 0.05% to 10% by weight, and such as from 0.5% to 10% by weight, relative to the total weight of the final composition.

The ratio by weight of the at least one cationic polymer (i) according to the present disclosure described above to the at least one anionic associative polymer (ii) ranges from 0.1 to 10, such as from 0.5 to 5.

The compositions according to the present disclosure may also contain at least one surfactant including those chosen from anionic, amphoteric, nonionic, and cationic surfactants.

Non-limiting examples of the at least one anionic surfactant include alkaline metal salts, ammonium salts, amine salts, amino alcohol salts, and magnesium salts of the following compounds: alkyl sulfates, alkyl ether sulfates, alkylamido ether sulfates, alkylarylpolyether sulfates, monoglyceride sulfates; alkyl sulfonates, alkylamide sulfates, alkylaryl sulfonates, olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl phosphates, alkyl ether phosphates; and acylsarcosinates, acylisethionates, and N-acyltaurates.

The alkyl or acyl radical of these various compounds may be constituted of a carbon-based chain containing from 8 to 30 carbon atoms.

Exemplary anionic surfactants include fatty acid salts, such as oleic acid, ricinoleic acid, palmitic acid, or stearic acid salts; coconut oil or hydrogenated coconut oil acids; and acyl lactylates, the acyl radical of which contains from 8 to 30 carbon atoms.

Use may also be made of surfactants considered to be weakly anionic, such as polyoxyalkylenated carboxylic alkyl or alkylaryl ether acids or salts thereof, polyoxyalkylenated carboxylic alkylamido ether acids or salts thereof, and alkyl D-galactosiduronic acids or salts thereof.

The nonionic surfactants may be chosen from polyethoxylated, polypropoxylated fatty acids, polyglycerolated fatty acids, alkylphenols, and alcohols, with a fatty chain containing from 8 to 30 carbon atoms, the number of ethylene oxide or propylene oxide groups ranging from 2 to 50, and the number of glycerol groups ranging from 2 to 30.

Non-limiting mention may also be made of copolymers of ethylene oxide and of propylene oxide; condensates of ethylene oxide and of propylene oxide with fatty alcohols; polyethoxylated fatty amides containing from 2 mol to 30 mol of ethylene oxide; polyglycerolated fatty amides comprising from 1 to 5 glycerol groups, such as from 1.5 to 4; polyethoxylated fatty amines containing from 2 mol to 30 mot of ethylene oxide; oxyethylenated sorbitan fatty acid esters containing from 2 mol to 30 mol of ethylene oxide; sucrose fatty acid esters, polyethylene glycol fatty acid esters, alkylpolyglycosides, carbamate or amide derivatives of N-alkylglucamines, aldobionamides, and amine oxides such as alkylamine oxides or N-acylamidopropylmorpholine oxides.

Exemplary amphoteric surfactants include secondary or tertiary aliphatic amine derivatives, wherein the aliphatic radical is a linear or branched chain containing from 8 to 22 carbon atoms, and which contains at least one carboxylate, sulfonate, sulfate, phosphate, or phosphonate water-solubilizing anionic group; such as (C₈-C₂₀)alkylbetaines, sulfobetaines, (C₈-C₂₀)alkylamido(C₁-C₆)alkylbetaines, or (C₈-C₂₀)alkylamido(C₁-C₆)alkylsulfobetaines.

Non-limiting examples of the amine derivatives include the products sold under the name Miranol, as described in U.S. Pat. Nos. 2,528,378 and 2,781,354 and classified in the CTFA dictionary, 7th edition, 1997, under the name Disodium Cocoamphodiacetate, Disodium Lauroamphodiacetate, Disodium Capryloamphodiacetate, Disodium Caproamphodiacetate, Disodium Cocoamphodipropionate, Disodium Lauroamphodipropionate, Disodium Caproamphodipropionate, Disodium Caprylo-amphodipropionate, Lauroamphodipropionate acid and Cocoamphodipropionate acid.

The at least one cationic surfactant may be chosen from optionally polyoxyalkylenated primary, secondary, and tertiary fatty amine salts; quaternary ammonium salts; imidazoline derivatives; and amine oxides of cationic nature.

Non-limiting examples of quaternary ammonium salts include tetraalkylammonium halides (for example chlorides) such as, for example, dialkyldimethylammonium or alkyltrimethylammonium chlorides, wherein the alkyl radical contains from 12 to 22 carbon atoms, such as behenyltrimethylammonium, distearyidimethylammonium, cetyltrimethylammonium, or benzyldimethylstearylammonium chloride, or alternatively stearamidopropyldimethyl(myristyl acetate)ammonium chloride sold under the name “Cepharyl 70” by the company Van Dyk.

Use may also be made of diacyloxyethyldimethylammonium, diacyloxyethylhydroxyethylmethylammonium, monoacyloxyethyl-dihydroxyethylmethylammonium, triacyloxyethylmethylammonium and monoacyloxyethyl-hydroxyethyldimethylammonium salts (chlorides or methylsulfate in particular), and mixtures thereof. The acyl radicals may contain from 14 to 18 carbon atoms, and may originate from a plant oil, such as palm oil or sunflower oil.

The surfactants are optionally used in the compositions in accordance with the present disclosure in amounts ranging from 0.01% to 50% by weight, relative to the total weight of the composition. When the compositions are in the form of shampoos, they are used in an amount of at least 4% by weight, such as from 5% to 50% by weight, relative to the total weight of the composition, even such as from 8% to 35%.

The compositions according to the present disclosure have a pH ranging from 3 to 12, such as from 4 to 8, and even such as from 4 to 6.

In an exemplary embodiment of the present disclosure, the cosmetic compositions may also contain conditioning agents for keratin materials.

When the composition contains at least one conditioning agent, it may be chosen from synthetic oils, such as poly-α-olefins, fluoro oils, fluoro waxes, fluoro gums, carboxylic acid esters, cationic polymers other than those of the present disclosure, silicones, mineral, plant, and animal oils, ceramides, and pseudoceramides.

The polyolefins may be poly-α-olefins, including:

hydrogenated or nonhydrogenated polybutene type, such as hydrogenated or nonhydrogenated polyisobutene type.

Use can be made of oligomers of isobutylene with a molecular weight of less than 1000, and mixtures thereof with polyisobutylenes with a molecular weight of greater than 1000, such as ranging from 1000 to 15 000.

By way of examples of poly-α-olefins that can be used in the context of the present disclosure, non-limiting mention may be made of the polyisobutenes sold under the name Permethyl 99 A, 101 A, 102 A, 104 A (n=16) and 106 A (n=38) by the company Presperse Inc., or else the products sold under the name Arlamol HD (n=3) by the company ICI (n denoting the degree of polymerization);

hydrogenated or nonhydrogenated polydecene type.

Such products are sold, for example, under the names Ethylflo by the company Ethyl Corp., and Arlamol PAO by the company ICI.

The mineral oils that can be used in the compositions of the present disclosure may be chosen from hydrocarbons, such as hexadecane and liquid paraffin.

The additional cationic polymers that can be used in accordance with the present disclosure may be chosen from all those known in the art, including those described in Patent Application EP-A-0 337 354 and in French Patent Applications FR-A-2 270 846, 2 383 660, 2 598 611, 2 470 596 and 2 519 863.

The cationic polymers used may have a molecular weight ranging from 500 to 5×10⁶ approximately, such as from 103 to 3×10⁶.

Exemplary cationic polymers include, but are not limited to, quaternized proteins (or protein hydrolysates) and polymers of the polyamine, polyaminoamide and polyquaternary ammonium type.

The quaternized protein or protein hydrolysates are chemically modified polypeptides bearing quaternary ammonium groups at the end of the chain or grafted onto said chain. Their molecular weight can range, for example, from 1500 to 10 000, such as from 2000 to 5000. Non-limiting examples include the following:

hydrolysates of collagen bearing triethylammonium groups, such as the products called, in the CTFA dictionary, “Triethonium Hydrolyzed Collagen Ethosulfate”;

hydrolysates of collagen bearing trimethylammonium chloride and trimethylstearylammonium chloride groups, called, in the CTFA dictionary, “Steartrimonium Hydrolyzed Collagen”; and

hydrolysates of proteins bearing, on the polypeptide chain, quaternary ammonium groups comprising at least one alkyl radical containing from 1 to 18 carbon atoms.

Among these protein hydrolysates, non-limiting mention may be made, inter alia, of “Croquat L”, “Croquat M”, “Croquat S” and “Crotein Q” sold by the company Croda.

Other quaternized proteins or hydrolysates are, for example, those sold by the company Inolex under the name “Lexein OX 3000”.

Non-limiting mention may also be made of quaternized plant proteins, such as wheat, corn or soybean proteins; as quaternized wheat proteins, non-limiting mention may be made of those called, in the CTFA dictionary, “Cocodimonium Hydrolysed Wheat Protein”, “Lauridimonium Hydrolysed Wheat Protein”, or else “Steardimonium Hydrolysed Wheat Protein”.

The polymers of the polyamine, polyamidoamide, or polyquaternary ammonium type that can be used in accordance with the present disclosure are those described, for example, in French Patents 2 505 348 and 2 542 997. Non-limiting examples of these polymers include:

(1) Homopolymers and copolymers derived from acrylic or methacrylic esters or amides and comprising at least one of the units of the following formulae:

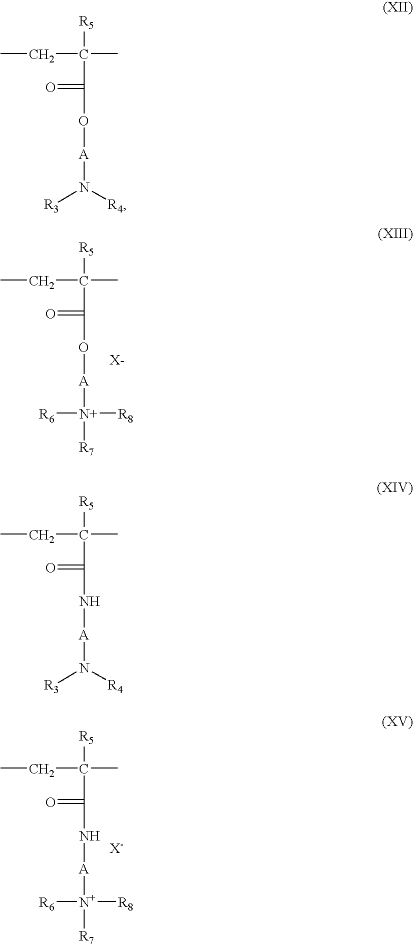

wherein:

R₃ and R₄, which may be identical or different, are a hydrogen atom or an alkyl group containing from 1 to 6 carbon atoms, such as methyl or ethyl;

R₅, which may be identical or different, is a hydrogen atom or a CH₃ radical;

A, which may be identical or different, is a linear or branched alkyl group containing from 1 to 6 carbon atoms, such as from 2 to 3 carbon atoms, or a hydroxyalkyl group containing from 1 to 4 carbon atoms;

R₆, R₇ and R₈, which may be identical or different, are an alkyl group containing from 1 to 18 carbon atoms or a benzyl radical, such as an alkyl group containing from 1 to 6 carbon atoms; and

X⁻ is an anion derived from a mineral or organic acid, such as a methosulfate anion, or a halide such as chloride or bromide.

The copolymers of family (1) may also contain at least one unit derived from comonomers that may be chosen from the family of acrylamides, methacrylamides, diacetone acrylamides, acrylamides, and methacrylamides substituted on the nitrogen with (C₁-C₄) lower alkyls, acrylic or methacrylic acids, and esters thereof, vinyllactams, such as vinylpyrrolidone or vinylcaprolactam, and vinyl esters.

Thus, among these copolymers of family (1), non-limiting mention may be made of:

copolymers of acrylamide and of dimethylaminoethyl methacrylate quaternized with dimethyl sulfate or with a dimethyl halide, such as that sold under the name Hercofloc by the company Hercules,

copolymers of acrylamide and of methacryloyloxyethyltrimethylammonium chloride described, for example, in Patent Application EP-A-080 976 and sold under the name Binaquat P 100 by the company Ciba Geigy,

the copolymers of acrylamide and of methacryloyloxyethyltrimethylammonium methosulfate sold under the name Reten by the company Hercules,

quaternized or nonquaternized vinylpyrrolidone/dialkylaminoalkyl acrylate or methacrylate copolymers, such as the product sold under the name “Gafquat” by the company ISP, for instance “Gafquat 734” or “Gafquat 755” or else the products known as “Copolymer 845, 958 and 937”. These polymers are described in detail in French Patents 2 077 143 and 2 393 573,

dimethylaminoethyl methacrylate/vinylcaprolactam/vinylpyrrolidone terpolymers, such as the product sold under the name Gaffix VC 713 by the company ISP,

vinylpyrrolidone/methacrylam idopropyldimethylamine copolymers sold in particular under the name Styleze CC 10 by ISP,

quaternized vinylpyrrolidone/dimethylaminopropylmethacrylamide copolymers, such as the product sold under the name “Gafquat HS 100” by the company ISP, and

crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, in particular methylenebisacrylamide. Use may be made of a crosslinked acrylamide/methacryloyloxyethyltrimethylammonium chloride copolymer (20/80 by weight) in the form of a dispersion containing 50% by weight of said copolymer in mineral oil. This dispersion is sold under the name “Salcare® SC 92” by the company Ciba. Use may also be made of a crosslinked methacryloyloxyethyltrimethylammonium chloride homopolymer containing approximately 50% by weight of the homopolymer in mineral oil or in a liquid ester. These dispersions are sold under the names “Salcare® SC 95” and “Salcare® SC 96” by the company Ciba;

(2) polymers constituted of piperazinyl units and of divalent alkylene or hydroxyalkylene radicals having straight or branched chains, optionally interrupted with oxygen, sulfur, or nitrogen atoms or with aromatic or heterocyclic rings, and also the products of oxidation and/or quaternization of these polymers. Such polymers are in particular described in French Patents 2 162 025 and 2 280 361;

(3) water-soluble polyaminoamides prepared in particular by polycondensation of an acid compound with a polyamine; these polyaminoamides may be crosslinked with an epihalohydrin, a diepoxide, a dianhydride, an unsaturated dianhydride, a bisunsaturated derivative, a bishalohydrin, a bisazetidinium, a bishaloacyidiamine, or a bis(alkyl halide), or else with an oligomer resulting from the reaction of a bifunctional compound which is reactive with respect to a bishalohydrin, a bisazetidinium, a bishaloacyidiamine, a bis(alkyl halide), an epilhalohydrin, a diepoxide or a bisunsaturated derivative; the crosslinking agent being used in proportions ranging from 0.025 mol to 0.35 mol per amine group of the polyaminoamide; these polyaminoamides may be alkoylated or, if they comprise at least one tertiary amine function, quaternized. Such polymers are described in French Patents 2 252 840 and 2 368 508;

(4) polyaminoamide derivatives resulting from the condensation of polyalkylenepolyamines with polycarboxylic acids, followed by alkylation with bifunctional agents. Non-limiting mention may, for example, be made of adipic acid/dialkylaminohydroxyalkyldialkylenetriamine polymers wherein the alkyl radical contains from 1 to 4 carbon atoms, such as methyl, ethyl, or propyl. Such polymers are described in French Patent 1 583 363.

Among these derivatives, non-limiting mention may be made of the adipic acid/dimethylaminohydroxypropyldiethylenetriamine polymers sold under the name “Cartaretin F, F4 or F8” by the company Sandoz;

(5) polymers obtained by reaction of a polyalkylenepolyamine comprising two primary amine groups and at least one secondary amine group with a dicarboxylic acid chosen from diglycolic acid and saturated aliphatic dicarboxylic acids containing from 3 to 8 carbon atoms. The molar ratio of the polyalkylenepolyamine to the dicarboxylic acid ranges from 0.8:1 to 1.4:1; the polyaminoamide resulting from this reaction being reacted with epichlorohydrin in a molar ratio of epichlorohydrin relative to the secondary amine group of the polyaminoamide ranging from 0.5:1 to 1.8:1. Such polymers are described in U.S. Pat. Nos. 3,227,615 and 2,961,347.

Polymers of this type are sold under the name “Hercosett 57” by the company Hercules Inc. or else under the name “PD 170” or “Delsette 101” by the company Hercules in the case of the adipic acid/epoxypropyldiethylenetriamine copolymer;

(6) alkyldiallylamine or dialkyldiallylammonium cyclopolymers, such as homopolymers or copolymers comprising, as main constituent of the chain, units corresponding to formula (XVI) or (XVII):

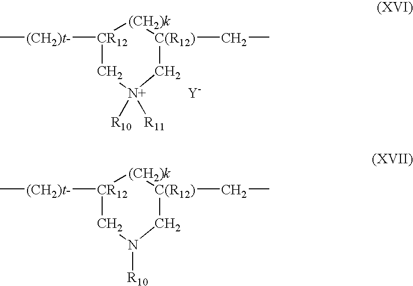

wherein k and t are equal to 0 or 1, the sum k+t being equal to 1; R₁₂ is a hydrogen atom or a methyl radical; R₁₀ and R₁₁, independently of one another, are an alkyl group containing from 1 to 6 carbon atoms, a hydroxyalkyl group wherein the alkyl group contains from 1 to 5 carbon atoms, or a (C₁-C₄) lower amidoalkyl group, or R₁₀ and R₁₁ may be, together with the nitrogen atom to which they are attached, heterocyclic groups, such as piperidinyl or morpholinyl; Y— is an anion such as bromide, chloride, acetate, borate, citrate, tartrate, bisulfate, bisulfite, sulfate, or phosphate. These polymers are described in French Patent 2 080 759 and in its certificate of addition 2 190 406.

R₁₀ and R₁₁, independently of one another, are an alkyl group containing from 1 to 4 carbon atoms.

Among the polymers defined above, non-limiting mention may be made of the dimethyldiallylammonium chloride homopolymer sold under the name “Merquat 100” by the company Nalco (and its homologues with low weight-average molecular weights) and the copolymers of diallyldimethylammonium chloride and of acrylamide sold under the name “Merquat 550”;

(7) the diquaternary ammonium polymer comprising repeat units corresponding to the formula.

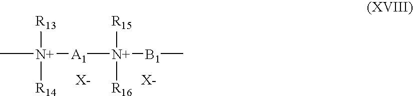

wherein formula (XVIII):

R₁₃, R₁₄, R₁₅ and R₁₆, which may be identical or different, are aliphatic, alicyclic, or arylaliphatic radicals containing from 1 to 20 carbon atoms or lower hydroxyalkylaliphatic radicals, or else R₁₃, R₁₄, R₁₅ and R₁₆, together or separately, constitute, with the nitrogen atoms to which they are attached, heterocycles optionally containing a second heteroatom other than nitrogen, or else R₁₃, R₁₄, R₁₅ and R₁₆ are linear or branched C₁-C₆ alkyl radicals substituted with a nitrile, ester, acyl, amide, —CO—O—R₁₇-D or —CO—NH—R₁₇-D group, where R₁₇ is an alkylene and D a quaternary ammonium group;

A₁ and B₁ are polymethylene groups containing from 2 to 20 carbon atoms that may be linear or branched, saturated or unsaturated, and that may contain, linked to or intercalated in the main chain, at least one aromatic ring, or at least one oxygen or sulfur atom or sulfoxide, sulfone, disulfide, amino, alkylamino, hydroxyl, quaternary ammonium, ureido, amide, or ester group, and

X⁻ is an anion derived from a mineral or organic acid;

A₁, R₁₃ and R₁₅ can form, with the two nitrogen atoms to which they are attached, a piperazine ring; in addition, if A₁ is a saturated or unsaturated, linear or branched alkylene or hydroxyalkylene radical, B₁ can also be a (CH₂)ₙₚ—CO-D-OC—(CH₂)p-group, where p is an integer ranging from 2 to 20, and wherein D is:

a) a glycol residue of formula: —O-Z-O—, where Z is a linear or branched hydrocarbon-based radical or a group corresponding to one of the following formulae:

—(CH₂—CH₂—O)ₓ—CH₂-CH₂—

—[CH₂—CH(CH₃)—O]_{y}—CH₂—CH(CH₃)—

wherein x and y are an integer from 1 to 4, representing a defined and unique degree of polymerization, or any number from 1 to 4, representing a mean degree of polymerization;

b) a bissecondary diamine residue such as a piperazine derivative;

c) a bisprimary diamine residue of formula: —NH—Y—NH—, wherein Y is a linear or branched hydrocarbon-based radical, or else the divalent radical

—CH₂—CH₂—S—S—CH₂—CH₂—; and

d) a ureylene group of formula: —NH—CO—NH—.

In at least one embodiment, X⁻ is an anion, such as chloride or bromide.

These polymers have a number-average molecular weight of ranging from 1000 to 100 000.

Polymers of this type are described in French Patents 2 320 330, 2 270 846, 2 316 271, 2 336 434 and 2 413 907, and U.S. Pat. Nos. 2,273,780; 2,375,853; 2,388,614; 2,454,547′ 3,206,462; 2,261,002; 2,271,378; 3,874,870; 4,001,432; 3,929,990; 3,966,904; 4,005,193; 4,025,617; 4,025,627; 4,025,653; 4,026,945; and 4,027,020.

Use may be made of the polymers which are constituted of repeat units corresponding to the formula:

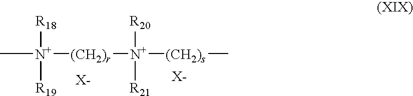

wherein R₁₈, R₁₉, R₂₀ and R₂₁, which may be identical or different, are chosen from an alkyl and a hydroxyalkyl radical containing from 1 to 4 carbon atoms, r and s are integers ranging from 2 to 20, and X— is an anion derived from a mineral or organic acid.

In an exemplary embodiment, the compound of formula (XIX) is that for which R₁₈, R₁₉, R₂₀ and R₂₁ are methyl radicals and r=3, s=6, and X—=Cl, known as hexadimethrine chloride according to INCI nomenclature (CTFA);

(8) polyquaternary ammonium polymers constituted of units of formula (XX):

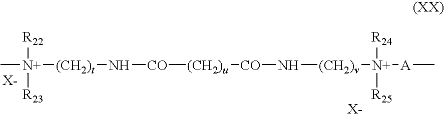

wherein R₂₂, R₂₃, R₂₄ and R₂₅, which may be identical or different, are chosen from a hydrogen atom or a methyl, ethyl, propyl, β-hydroxyethyl, β-hydroxypropyl, and —CH₂CH₂(OCH₂CH₂)ₚOH radical,
wherein p is equal to 0 or to an integer from 1 to 6, with the proviso that R₂₂, R₂₃, R₂₄ and
R₂₅ do not simultaneously represent a hydrogen atom,
t and u, which may be identical or different, are integers from 1 to 6,
v is equal to 0 or is an integer from 1 to 34,
X⁻ is an anion, such as a halide, and
A is a radical of a dihalide or is —CH₂—CH₂—O—CH₂—CH₂—.
Such compounds are described in Patent Application EP-A-122 324. Mention may, for example, be made, among these, of the products Mirapol® A 15, Mirapol® AD1, Mirapol® AZ1 and Mirapol® 175 sold by the company Miranol;

(9) Quaternary polymers of vinylpyrrolidone and of vinylimidazole, such as, for example, the products sold under the names Luviquat FC 905, FC 550 and FC 370 by the company BASF; and

(10) Cationic polysaccharides, such as celluloses and gums of cationic galactomannans. Among the cationic polysaccharides, non-limiting mention may be made of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups are described in French Patent 1 492 597, and in particular the polymers sold under the names JR (JR 400, JR 125, JR 30M) or LR (LR 400, LR 30M) by the company Nalco. These polymers are also defined in the CTFA dictionary as quaternary ammoniums of hydroxyethylcellulose having reacted with an epoxide substituted with a trimethylammonium group.

The cationic cellulose copolymers or the cellulose derivatives grafted with a water-soluble quaternary ammonium monomer are described in U.S. Pat. No. 4,131,576, such as hydroxyalkylcelluloses, for instance hydroxymethyl-, hydroxyethyl- or hydroxypropylcelluloses grafted with a methacryloylethyltrimethylammonium, methacrylamidopropyltrimethylammonium, or dimethyldiallylammonium salt.

The commercial products corresponding to this definition include the products sold under the names Celquat L 200 and Celquat H 100 by the company National Starch.

The cationic galactomannan gums are described more particularly in U.S. Pat. Nos. 3,589,578 and 4,031,307, such as guar gums containing trialkylammonium cationic groups. Guar gums modified with a 2,3-epoxypropyltrimethylammonium salt (for example chloride) are, for example, used.

Such products are sold under the trade names Jaguar C13S, Jaguar C15, Jaguar C 17, Jaguar Excel and Jaguar C162 by the company Rhodia Chimie.

Other cationic polymers that can be used in the context of the present disclosure are cationic proteins or cationic protein hydrolysates, polyalkyleneimines, such as polyethyleneimines, polymers containing vinylpyridine or vinylpyridinium units, condensates of polyamines and of epichlorohydrin, polyquaternary ureylenes and chitin derivatives.

Among all the cationic polymers that can be employed in the context of the present disclosure, use may be made of quaternary cellulose ether derivatives, such as the products sold under the name “JR 400” by the company Amerchol, cyclopolymers, such as homopolymers of a diallyidimethylammonium salt and copolymers of a diallyidimethylammonium salt and of acrylamide, such as the chlorides, sold under the names “Merquat 550” and “Merquat S” by the company Nalco, cationic polysaccharides, and the guar gums modified with 2,3-epoxypropyltrimethylammonium chloride sold, for example, under the name “Jaguar C13S” by the company Rhodia Chimie, optionally crosslinked homopolymers and copolymers of a (meth)acryloyloxyethyltrimethylammonium salt, sold by the company Ciba, in solution at 50% in mineral oil, under the trade names Salcare SC92 (crosslinked copolymer of methacryloyloxyethyltrimethylammonium chloride and of acrylamide) and Salcare SC95 (crosslinked homopolymer of methacryloyloxyethyltrimethylammonium chloride), quaternary copolymers of vinylpyrrolidone and of a vinylimidazole salt, such as the products sold by BASF under the names Luviquat FC 370, Luviquat FC 550, Luviquat FC 905, and Luviquat HM-552.

The silicones that may be used in accordance with the present disclosure are polyorganosiloxanes that are insoluble in the composition and may be in the form of oils, waxes, resins, or gums.

In an exemplary embodiment of the present disclosure, the compositions according to the present disclosure also comprise at least one silicone, such as modified or unmodified polyorganosiloxanes, for example polyorganosiloxane oils or polyorganosiloxane gums or resins, as they are, or in the form of solutions in organic solvents or else in the form of emulsions or microemulsions.

Among the polyorganosiloxanes that can be used in accordance with the present disclosure, non-limiting mention may be made of:

I. Volatile silicones: these have a boiling point ranging from 60° C. to 260° C. They are chosen from cyclic silicones containing from 3 to 7 silicon atoms, such as from 4 to 5. They are, for example, the octamethylcyclotetrasiloxane sold under the name “Volatile Silicone 7207” by Union Carbide or “Silbione 70045 V2” by Rhone Poulenc, the decamethylcyclopentasiloxane sold under the name “Volatile Silicone 7158” by Union Carbide and “Silbione 70045 V5” by Rhone Poulenc, and mixtures thereof. Non-limiting mention is also made of cyclocopolymers of the dimethylsiloxane/methylalkylsiloxane type, such as the “Silicone Volatile FZ 3109” sold by the company Union Carbide, which is a dimethylsiloxane/methyloctylsiloxane cyclocopolymer.

II. Nonvolatile silicones: they are mainly constituted of:

(i) polyalkylsiloxanes; among the polyalkylsiloxanes, non-limiting mention may be made of linear polydimethylsiloxanes having trimethylsilyl end groups, for instance, the “Silbione” oils of the 70047 series sold by Rhodia Chimie; the DC 200 oils from Dow Corning, and PDMSs having hydroxydimethylsilyl end groups;

(ii) polyarylsiloxanes;

(iii) polyalkylarylsiloxanes; non-limiting mention may be made of linear and branched polymethylphenylsiloxanes, polydimethylmethylphenylsiloxanes, and polydimethyldiphenylsiloxanes, such as, for example, the oil “Rhodorsil 70763” from Rhodia Chimie;

(iv) silicone gums; these are polydiorganosiloxanes having a molecular weight of ranging from 200 000 to 5 000 000, used alone or as a mixture in a solvent chosen from volatile silicones, polydimethylsiloxane (PDMS) oils, polyphenylmethylsiloxane (PPMS) oils, isoparaffins, methylene chloride, pentane, dodecane, tridecane, and tetradecane; for example:
polydimethylsiloxane gums,
poly[(dimethylsiloxane)/(methylvinyisiloxane)] gums
poly[(dimethylsiloxane)/(diphenylsiloxane)] gums,
poly[(dimethylsiloxane)/(phenylmethylsiloxane)] gums, and
poly[(dimethylsiloxane)/(diphenylsiloxane)/(methylvinylsiloxane)] gums.

Non-limiting mention may also be made, for example, of the following mixtures:

1) mixtures formed from a polydimethylsiloxane gum hydroxylated at the end of the chain (Dimethiconol according to the CTFA nomenclature), and from a cyclic polydimethylsiloxane (Cyclomethicone according to the CTFA nomenclature), such as the product “Q2 1401” sold by the company Dow Corning;

2) mixtures formed from a polydimethylsiloxane gum with a cyclic silicone, such as the product “SF 1214 Silicone Fluid” from General Electric, which is an SE 30 gum of MW 500 000 solubilized in “SF 1202 Silicone Fluid” (decamethylcyclopentasiloxane);

3) mixtures of two PDMSs of different viscosity, such as of a PDMS gum and of a PDMS oil, for example the products “SF 1236” and “CF 1241” from the company General Electric;

(v) silicone resins: crosslinked siloxane systems containing R₂SiO_{2/2}, RSiO_{3/2} and SiO_{4/2} units wherein R is a hydrocarbon-based group having from 1 to 6 carbon atoms or a phenyl group. Among these resins, non-limiting mention may be made of the product sold under the name “Dow Corning 593”;

(vi) organomodified polyorganosiloxanes; such as silicones as defined above, comprising, in their general structure at least one organofunctional group directly attached to the siloxane chain or attached by means of a hydrocarbon-based radical; non-limiting mention is, for example, made of silicones comprising:

a) polyethyleneoxy and/or polypropyleneoxy groups, optionally comprising alkyl groups, such as the product known as dimethicone copolyol sold by the company Dow Corning under the name “DC 1248”, and the alkyl (C12) methicone copolyol sold by the company Dow Corning under the name “Q2 5200”;

b) (per)fluoro groups, for instance trifluoroalkyl groups, such as, for example, those sold by the company General Electric under the name “FF.150 Fluorosilicone Fluid”;

c) hydroxyacylamino groups, such as those described in European Patent

Application EP-A-0 342 834, and in particular the silicone sold by the company Dow Corning under the name “Q2-8413”;

d) thiol groups, such as the silicones “X 2-8360” from Dow Corning or “GP 72A” and “GP 71” from Genesee;

e) substituted or unsubstituted amino groups, such as the products sold under the name GP 4 Silicone Fluid and GP 7100 by the company Genesee or the products sold under the names O2 8220 and Dow Corning 929 or 939 by the company Dow Corning. The substituted amino groups may be C₁-C₄ aminoalkyl or amino(C₁-C₄)alkylamino(C₁-C₄)alkyl groups. The silicones known as amodimethicone and trimethylsilylamodimethicone according to the CTFA name (1997) may be used;

f) carboxylate groups, such as the products described in European Patent EP 186 507 from Chisso Corporation;

g) hydroxylated groups, such as the polyorganosiloxanes comprising a hydroxyalkyl function, described in Patent Application FR-A-2 589 476;

h) alkoxylated groups comprising at least 12 carbon atoms, such as the product “Silicone Copolymer F 755” from SWS Silicones;

i) acyloxyalkyl groups containing at least 12 carbon atoms, such as the polyorganosiloxanes described in Patent Application FR-A-2 641 185;

j) quaternary ammonium groups, such as the product “Abil K 3270” from the company Goldschmidt;

k) amphoteric or betaine groups, such as in the product sold by the company Goldschmidt under the name “Abil B 9950”; and

l) bisulfite groups, such as in the products sold by the company Goldschmidt under the names “Abil S 201” and “Abil S 255”; vii) block copolymers having a polysiloxane-polyalkylene linear block as repeat unit; the preparation of such block copolymers used in the context of the present disclosure is described in European Application EP 0 492 657 A1, the teaching of which is included in the present description by way of reference;

viii) grafted silicone polymers, having a nonsilicone organic backbone constituted of a main organic chain formed from organic monomers which do not comprise silicone, onto which is grafted, within said chain and also, optionally, at least one of its ends, at least one polysiloxane macromonomer; such as those chosen from those described in U.S. Pat. Nos. 4,963,935; 4,728,571; and 4,972,037 and Patent Applications EP-A-0 412 704, EP-A-0 412 707, EP-A-0 640 105 and WO 95/00578, the teachings of which are entirely included in the present description by way of non-limiting references;

(ix) grafted silicone polymers, having a polysiloxane backbone grafted with nonsilicone organic monomers, comprising a polysiloxane main chain onto which is grafted, within said chain and also, optionally, at least one of its ends, at least one organic macromonomer which does not comprise silicone; examples of such polymers, and also the particular method for preparing them, are described, for example, in Patent Applications EP-A-0 582 152, WO 93/23009, and WO 95/03776, the teachings of which are entirely included in the present description by way of non-limiting references; and

(x) or mixtures thereof.

Exemplary silicones for use according to the present disclosure are non-volatile polyorganopolysiloxanes, such as polydimethylsiloxane oils or gums which optionally comprise amino, aryl, or alkylaryl groups.

The silicones are used in the compositions of the present disclosure in amounts ranging from 0.01% to 20% by weight, such as from 0.1% to 10% by weight, relative to the total weight of the composition.

According to the present disclosure, the compounds of ceramide type are, for example, natural or synthetic ceramides and/or glycoceramides and/or pseudoceramides and/or neoceramides.

Compounds of ceramide type are, for example, described in Patent Applications DE442-4530, DE442-4533, DE4402929, DE4420736, WO 95/23807, WO 94/07844, EP-A-0646572, WO 95/16665, FR-2-673 179, EP-A-0227994, WO 94/07844, WO 94/24087 and WO 94/10131, the teachings of which are included herein by way of reference.

Exemplary compounds of ceramide type according to the present disclosure are
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol, such as
N-stearoylphytosphingosine,
2-N-palmitoylaminohexadecane-1,3-diol,
(bis(N-hydroxyethyl-N-cetyl)malonamide),
cetyl acid N-(2-hydroxyethyl)-N-(3-cetyloxy-2-hydroxypropyl)amide,
N-docosanoyl-N-methyl-D-glucamine,

and mixtures of these compounds.

The liquid fatty esters that can be used in the present disclosure may be monomeric, such as nonionic, nonsilicone esters.

According to the present disclosure, the term “liquid fatty esters” means an ester that is liquid at room temperature (25° C.) and at atmospheric pressure (1 atm) and which is derived from a carboxylic acid and an alcohol, at least one of these two constituents containing at least 7 carbon atoms.

The liquid fatty esters according to the present disclosure are chosen from:

1) esters of a C₃-C₃₀ carboxylic acid and of a C₁-C₃₀ alcohol, at least one from among the acid and the alcohol being branched or unsaturated (containing at least one carbon-carbon double bond), and

2) esters of a C₇-C₃₀ aromatic acid whose carboxylic function is directly connected to be aromatic ring, and of a C₁-C₃₀ alcohol.

The esters according to the disclosure may be water-insoluble liquid esters.

The water-insoluble liquid carboxylic acid esters are insoluble in water at a concentration of greater than or equal to 0.1% by weight in water at 25° C., that is they do not form, under these conditions, a macroscopically isotropic transparent solution.

The total carbon number of the esters of the present disclosure is, for example, greater than or equal to 10, and less than 50, such as less than 25.

The liquid esters of a C₃-C₃₀ carboxylic acid and of a C₁-G₃₀ alcohol, at least one from among the acid and the alcohol being branched or unsaturated, are chosen from esters of a C₆-C₂₄ carboxylic acid and of a C₃-C₂₀ alcohol.

The esters according to the present disclosure are chosen from:

esters of a linear carboxylic acid containing from 12 to 26 carbon atoms and of a branched alcohol containing from 3 to 12 carbon atoms,

esters of a linear carboxylic acid containing from 2 to 12 carbon atoms and of a branched alcohol containing from 8 to 26 carbon atoms, and

esters of a branched carboxylic acid containing from 8 to 26 carbon atoms, such as from 8 to 12, and of a branched alcohol containing from 8 to 26 carbon atoms, such as from 8 to 12.

Non-limiting mention may be made of octyldodecyl behenate; isocetyl behenate; isocetyl lactate; isostearyl lactate; linoleyl lactate; oleyl lactate; isostearyl octanoate; isocetyl octanoate; decyl oleate; isocetyl isostearate; isocetyl laurate; isocetyl stearate; isodecyl octanoate; isodecyl oleate; isononyl isononanoate; isostearyl paimitate; myristyl isostearate; octyl isononanoate; 2-ethylhexyl isononate; octyl isostearate; octyldodecyl erucate; isopropyl palmitate, 2-ethylhexyl palmitate, 2-octyidecyl palmitates, branched alkyl myristates such as isopropyl myristate, t-butyl myristate or 2-octyldodecyl myristate, hexyl isostearate, butyl isostearate, isobutyl stearate; and 2-hexyldecyl laurate.

Esters of C₄-C₂₂ dicarboxylic or tricarboxylic acids and of C₁-C₂₂ alcohols and the esters of monocarboxylic, dicarboxylic, or tricarboxylic acids and of C₂-C₂₆ dihydroxy, trihydroxy, tetrahydroxy, or pentahydroxy alcohols may also be used according to the present disclosure.

Non-limiting mention may be made of diisopropyl sebacate; diisopropyl adipate; diisostearyl adipate; octyldodecyl stearoyl stearate; pentaerythrityl tetraisononanoate; pentaerythrityl tetraisostearate; triisopropyl citrate; triisostearyl citrate; and trioctyldodecyl citrate.

Liquid esters of a branched carboxylic acid containing from 4 to 6 carbon atoms and of an alcohol containing from 8 to 26 carbon atoms may also be used.

These branched liquid esters according to the present disclosure have the following formula:

R₁COOR₂ (XXI)

wherein:

R₁ is an optionally monohydroxylated or polyhydroxylated, branched hydrocarbon-based radical containing from 3 to 5 carbon atoms,

R₂ is an optionally monohydroxylated or polyhydroxylated, linear or branched, hydrocarbon-based radical containing from 12 to 26 carbon atoms, such as from 16 to 22 carbon atoms.

In one embodiment, R₁ is a branched alkyl radical containing from 3 to 5 carbon atoms, such as a tert-butyl radical.

In one embodiment, R₂ is a saturated or unsaturated alkyl radical containing 12 to 26 carbon atoms, which may be branched, such as those chosen from tridecyl, isocetyl, isostearyl, octyldodecyl, and isoarachidyl radicals.

Exemplary branched liquid esters include, but are not limited to, isostearyl neopentanoate (formula (XXI) wherein R₁=tert-butyl and R₂=isostearyl), tridecyl neopentanoate, isocetyl neopentanoate, and isoarachidyl neopentanoate.

In at least one embodiment, the acid and the alcohol of the ester are saturated. In at least one embodiment, the alcohol is a monoalcohol (a single hydroxyl function).

Among the esters mentioned above, in at least one embodiment, the ester is chosen from isopropyl palmitate, 2-ethylhexyl palmitate, 2-octyldecyl palmitate, branched alkyl myristates, such as isopropyl myristate, t-butyl myristate, 2-octyidodecyl myristate, hexyl isostearate, butyl isostearate, isobutyl stearate, 2-hexyldecyl laurate, isostearyl neopentanoate, tridecyl neopentanoate, and isononyl isononanoate.

The esters of a C₇-C₃₀ aromatic acid and of a C₁-C₃₀ alcohol may be esters of a C₇-C₁₇ aromatic acid and of a C₁-C₂₀ alcohol. These esters include C₁₂-C₁₅ alkyl benzoates, isostearyl benzoate, octyldodecyl benzoate, behenyl benzoate, and 2-ethylhexyl benzoate.

According to the present disclosure, the conditioning agents may be present in an amount ranging from 0.001% to 10% by weight, such as from 0.005% to 5% by weight, such as from 0.01% to 3% by weight, relative to the total weight of the final composition.

The physiologically acceptable medium may be constituted of water or a mixture of water and cosmetically or dermatologically acceptable solvents such as monoalcohols, polyols or polyol ethers which can be used alone or as a mixture. The water may be present in an amount ranging from 30% to 98% by weight, such as from 50% to 98% by weight, relative to the total weight of the composition.

Exemplary monoalcohols include, but are not limited to, ethanol and isopropanol. Exemplary polyols include diethylene glycol and glycerol, and exemplary polyol ethers include diethylene glycol ethers.

The composition of the present disclosure may also contain at least one additive chosen from sequestering agents, emollients, foam modifiers, dyes, pearlescent agents, moisturizers, anti-dandruff or anti-seborrhoeic agents, suspending agents, fatty acids, thickeners, fragrances, preservatives, sunscreens, proteins, vitamins and provitamins, anionic non-associative polymers, nonionic or amphoteric polymers, sugars, menthol, nicotinate derivatives, hair-loss counteractants, foam stabilizers, propellants, vitamins or provitamins, and basifying or acidifying agents and any other additive conventionally used in the cosmetics field.

These additives are present in the composition according to the present disclosure in amounts ranging from 0 to 40% by weight relative to the total weight of the composition. The precise amount of each additive depends on its nature and is readily determined by those skilled in the art.

Those skilled in the art will take care to chose the possible compound(s) to be added to the composition according to the present disclosure in such a way that the desired properties intrinsically associated with the composition in accordance with the present disclosure are not, or not substantially, impaired by the addition envisaged.

The compositions in accordance with the present disclosure may be used for treating keratin materials such as hair, skin, eyelashes, eyebrows, nails, lips, and scalp.

The compositions may also be used for washing and cleansing keratin materials, such as hair and skin.

The compositions according to the present disclosure may be used as products for washing, caring for, conditioning, retaining the hairstyle, or shaping keratin materials, such as hair.

The compositions of the present disclosure may be in the form of shampoos, rinse-out or leave-in conditioners, compositions for permanent-waving, hair-straightening, dyeing or bleaching, or else in the form of compositions to be applied before or after a dyeing, bleaching, permanent-waving or hair-straightening operation, or alternatively between the two steps of a permanent-waving or hair-straightening operation.

In one embodiment, the compositions are washing and foaming compositions for hair and/or skin.

The compositions according to the present disclosure are foaming detergent compositions such as shampoos, shower gels, bubble baths, and makeup-removing products.

The minimum amount of surfactant is that which is just sufficient to confer on the final composition a satisfactory foaming and/or detergent power.

Thus, according to the present disclosure, the detergent surfactant may be present in an amount ranging from 3% to 30% by weight, such as from 6% to 25% by weight, such as from 8% to 20% by weight, relative to the total weight of the final composition.

The foaming power of the compositions according to the present disclosure, characterized by a foam height, is, for example, greater than 75 mm, such as greater than 100 mm, measured according to the modified Ross-Miles method (NF T 73-404/ISO696).

The modifications to the method are as follows:

The measurement is performed at a temperature of 22° C. with osmosed water. The concentration of the solution is 2 g/l. The drop height is 1 m. The amount of composition which is dropped is 200 ml. These 200 ml of composition fall into a measuring cylinder having a diameter of 50 mm and containing 50 ml of the test composition. The measurement is carried out 5 minutes after the flow of the composition has been stopped.

In another embodiment, when the composition is in the form of an optionally rinse-out conditioner, it may contain a cationic surfactant, the concentration of said surfactant generally ranging from 0.1% to 10% by weight, such as from 0.5% to 5% by weight, relative to the total weight of the composition.

The compositions according to the present disclosure may be in the form of aqueous or aqueous-alcoholic lotions for skin care and/or hair care.

The cosmetic compositions according to the present disclosure may be in the form of a gel, a milk, a cream, an emulsion, a thickened lotion, or a mousse and may be used for skin, nails, eyelashes, lips and hair.

The compositions may be packaged in various forms, such as in vaporizers, pump-dispenser bottles, or aerosol containers in order to be able to apply the composition in a vaporized form or in the form of a mousse. Such packaging forms are indicated, for example, when it is desired to obtain a spray, a lacquer, or a mousse for treating the hair.

An embodiment of the present disclosure is also a cosmetic treatment process for keratin materials such as hair, comprising applying a composition as defined above to the hair, and then optionally rinsing with water after an optional leave-in time.

The present disclosure will now be illustrated more fully with the aid of the following examples, which cannot be considered as limiting it to the embodiments described. In the subsequent text, AM signifies Active Material.

As disclosed herein, the percentages stated are by weight.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The examples which follow are intended to illustrate the present disclosure.

### EXAMPLES 1 to 3

The following shampoo composition was prepared:

| | 1 | | 2 | | 3 | |
|---|---|---|---|---|---|---|
| Sodium lauryl ether sulfate (2.2 EO) as | 10.5 | g AM | 10.5 | g AM | 10.5 | g AM |
| an aqueous solution containing 30% AM | | | | | | |
| (Texapon AOS 225 UP from Cognis) | | | | | | |
| Cocoamidopropylbetaine as an aqueous | 1.5 | g AM | 1.5 | g AM | 1.5 | g AM |
| solution containing 30% AM | | | | | | |
| (Tegobetaine F 50 from Goldschmidt) | | | | | | |
| Copolymer of acrylic or methacrylic acid | 1.2 | g AM | 1.6 | g AM | 2.1 | g AM |
| esters, of C1-4 dialkylamino C1-6 alkyl | | | | | | |
| methacrylate, of 30/5 PEG/PPG allyl | | | | | | |
| ether, of C10-30 20-25 PEG alkyl ether | | | | | | |
| methacrylate, of C2-6 hydroxyalkyl | | | | | | |
| methacrylate crosslinked with ethylene | | | | | | |
| glycol dimethacrylate, as a 20% | | | | | | |
| emulsion in water (Carbopol Aqua CC | | | | | | |
| from Noveon) | | | | | | |
| Steareth-20 acrylate and methacrylate | 1.35 | g AM | 1.35 | g AM | 1.35 | g AM |
| copolymers (Aculyn 22 Polymer from | | | | | | |
| Rohm & Haas) | | | | | | |
| Hydroxypropyl guar trimethylammonium | 0.1 | g | 0.1 | g | 0.1 | g |
| chloride (Jaguar Excel from Rhodia) | | | | | | |
| Olive oil | 1 | g | 1 | g | 1 | g |
| Dyes | 0.004 | g | 0.004 | g | 0.004 | g |
| Salicylic acid powder | 0.2 g | 0.2 | g | 0.2 | g | |
| Ethyl p-hydroxybenzoate | 0.15 | g | 0.15 | g | 0.15 | g |
| Sodium benzoate | 0.5 | g | 0.5 | g | 0.5 | g |
| Methyl p-hydroxybenzoate, sodium salt | 0.4 | g | 0.4 | g | 0.4 | g |
| Fragrance | 0.5 | g | 0.5 | g | 0.5 | g |
| Sodium hydroxide or lactic acid | qs pH 5.3 | | qs pH 5.3 | | qs pH 5.3 | |
| Deionized water qs | 100 | g | 100 | g | 100 | g |

These compositions were shampoos with a pleasant thickened texture. They were stable. When applied to the hair and then rinsed out, they gave the hair good disentangling, softness, smoothness, and sheen properties.

### EXAMPLE 4

The hair conditioner composition below was prepared:

| Polyacrylate-1 Crosspolymer as a 20% (by weight) | 1 | g AM |
|---|---|---|
| emulsion in water⁽¹⁾ | | |
| Methacrylic acid/ethyl acrylate/oxyethylenated (25 EO) | 0.1 | g AM |
| behenyl methacrylate terpolymer as an aqueous | | |
| emulsion at 20% by weight⁽²⁾ | | |
| Chlorhexidine digluconate solution | 0.2 | g |
| Methyl p-hydroxybenzoate | 0.3 | g |
| Lactic acid qs | pH 4.0 ± 0.2 | |
| Cetylstearyl alchol⁽³⁾ | 5 | g |
| Water qs | 100 | g |

⁽¹⁾sold under the trade name Carbopol Aqua CC Polymer by the company Noveon
⁽²⁾sold under the trade name Aculyn 28 by Rohm & Haas
⁽³⁾sold under the trade name Lanette O OR by the company Cognis

This hair conditioner composition was stable and thick. When applied to wet hair after shampooing, it was applied easily, spreading easily, and was then removed easily with water, leaving the hair smooth and shiny.

What is claimed is:

1. A cosmetic composition comprising, in a cosmetically acceptable medium:
(i) at least one cationic polymer which is produced by polymerization of a mixture of monomers comprising:
a) at least one vinyl monomer substituted with at least one amino group,
b) at least one hydrophobic nonionic vinyl monomer, chosen from a compound of formula (I) and (II):
CH₂═C(X)Z, and (I)
CH₂═CH—OC(O)R; (II)
wherein:
X is a hydrogen atom or a methyl group;
Z is chosen from —C(O)OR¹, —C(O)NH₂, —C(O)NHR¹, —C(O)N(R¹)₂, —C₆H₅, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R is independently a C₁-C₃₀ alkyl group;
each R¹ is independently chosen from a C₁-C₃₀ alkyl group, a hydroxylated C₂-C₃₀ alkyl group, and a halogenated C₁-C₃₀ alkyl group; and
c) at least one associative vinyl monomer,
e) at least one hydroxylated nonionic vinyl monomer; and
(ii) at least one anionic associative polymer.

2. The composition according to claim 1, wherein the at least one vinyl monomer substituted with at least one amino group is chosen from:
mono(C₁-C₄)alkylamino(C₁-C₈)alkyl (meth)acrylates,
di(C₁-C₄)alkylamino(C₁-C₈)alkyl (meth)acrylates, preferably di(C₁-C₄)alkylamino(C₁-C₆)alkyl (meth)acrylates,
mono(C₁-C₄)alkylamino(C₁-C₈)alkyl(meth)acrylamides,
di(C₁-C₄)alkylamino(C₁-C₈)alkyl(meth)acrylamides,
heterocyclic (meth)acrylamides containing a nitrogen atom, and
heterocyclic (meth)acrylates containing a nitrogen atom.

3. The composition according to claim 2, wherein the at least one vinyl monomer substituted with at least one amino group is chosen from:
mono- and di(C₁-C₄ alkyl)amino(C₁-C₄ alkyl) (meth)acrylates;
mono- and di(C₁-C₄ alkyl)amino(C₁-C₄ alkyl)(meth)acrylamides;
(meth)acrylamides and (meth)acrylates comprising a heterocyclic group containing a nitrogen atom; and
nitrogenous heterocycles comprising a vinyl group.

4. The composition according to claim 1, wherein in the at least one vinyl monomer substituted with at least one amino group is present in an amount ranging from 10% to 70% by weight, relative to the total weight of the mixture of monomers.

5. The composition according to claim 1, wherein the at least one hydrophobic nonionic vinyl monomer is chosen from C₁-C₃₀ alkyl (meth)acrylates, (C₁-C₃₀ alkyl)(meth)acrylamides, styrene, substituted styrenes, vinyl esters, unsaturated nitriles, and unsaturated silanes.

6. The composition according to claim 1, wherein the at least one hydrophobic nonionic vinyl monomer is present in an amount ranging from 20% to 80% by weight, relative to the total weight of the mixture of monomers.

7. The composition according to claim 1, wherein the at least one associative vinyl monomer is chosen from a compound of formula (III): 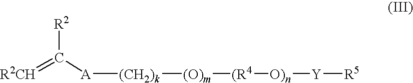
wherein
each R² is independently chosen from a hydrogen atom, a methyl group, a —C(O)OH, group and a —C(O)OR³ group;
R³ is a C₁-C₃₀ alkyl;
A is chosen from —CH₂C(O)O—, —C(O)O—, —O—, CH₂O, —NHC(O)NH—, —C(O)NH—, —Ar—(CE₂)_{z}—NHC(O)O—, —Ar—(CE₂)_{z}—NHC(O)NH—, and —CH₂CH₂—NHC(O)— groups;
Ar is a divalent aryl group;
E is a hydrogen atom or a methyl group;
z is equal to 0 or 1;
k is an integer ranging from 0 to 30;
m is equal to 0 or 1, with the proviso that, when k=0, m=0, and when k ranges from 1 to 30, m is equal to 1;
(R⁴—O), is a polyoxyalkylene, which is a homopolymer, a random copolymer, or a block copolymer, with C₂-C₄ oxyalkylene units;
R⁴ is chosen from C₂H₄, C₃H₆, and C₄H₈;
n is an integer ranging from 5 to 250;
Y is chosen from —R⁴⁰—, —R⁴NH—, —C(O)—, —C(O)NH—, R⁴NHC(O)NH—, and —C(O)NHC(O)—;
R⁵ is a substituted or unsubstituted alkyl chosen from linear C₈-C₄₀ alkyls, branched C₈-C₄₀ alkyls, C₈-C₄₀ alicyclics, phenyls substituted with a C₂-C₄₀ alkyl group, C₂-C₄₀ alkyls substituted with an aryl group, and C₈-C₈₀ complex esters, and
the R⁵ alkyl group optionally comprises at least one substituent chosen from hydroxyl, alkoxy, and halo groups.

8. The composition according to claim 7, wherein the at least one associative vinyl monomer is chosen from polyethoxylated cetyl (meth)acrylates, polyethoxylated cetearyl (meth)acrylates, polyethoxylated stearyl (meth)acrylates, polyethoxylated arachidyl (meth)acrylates, polyethoxylated behenyl (meth)acrylates, polyethoxylated lauryl (meth)acrylates, polyethoxylated cerotyl (meth)acrylates, polyethoxylated montanyl (meth)acrylates, polyethoxylated melissyl (meth)acrylates, polyethoxylated lacceryl (meth)acrylates, polyethoxylated 2,4,6-tri(1′-phenylethyl)phenyl (meth)acrylates, polyethoxylated (meth)acrylates of hydrogenated castor oil, polyethoxylated canola (meth)acrylates, and polyethoxylated (meth)acrylates of cholesterol, wherein the polyethoxylated portion of the monomer comprises from 5 to 100 ethylene oxide units.

9. The composition according to claim 1, wherein the at least one associative vinyl monomer is present in an amount ranging from 0.001% to 25% by weight, relative to the total weight of the mixture of monomers.

10. The composition according to claim 1, wherein the mixture of monomers comprises at least one semi-hydrophobic surfactant vinyl monomer d) chosen from compounds of formula (IV) and (V): 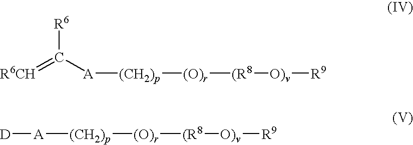
wherein,
each R⁶ is independently chosen from a hydrogen atom, a C₁-C₃₀ alkyl, —C(O)OH, and C(O)OR⁷;
R⁷ is a C₁-C₃₀ alkyl;
A is chosen from a —CH₂C(O)O—, —C(O)O—, —O—, —CH₂O, —NHC(O)NH—, —C(O)NH—, —Ar—(CE₂)_{z}—NHC(O)O—, —Ar—(CE₂)_{z}—NHC(O)NH—, and —CH₂CH₂NHC(O)— group;
Ar is a divalent aryl group;
E is a hydrogen atom or a methyl group;
z is equal to 0 or 1;
p is an integer ranging from 0 to 30;
r is equal to 0 or 1, with the proviso that, when p is equal to 0, r is equal to 0, and when p ranges from 1 to 30, r is equal to 1;
(R₈—O)ᵥ is a polyoxyalkylene which is a homopolymer, a random copolymer, or a block copolymer with C₂-C₄ oxyalkylene units, wherein R⁸ is chosen from C₂H₄, C₃H₆, and C₄H₈, and v is an integer ranging from 5 to 250;
R⁹ is a hydrogen atom or a C₁-C₄ alkyl; and
D is a C₈-C₃₀ alkenyl or a C₈-C₃₀ alkenyl substituted with a carboxyl group.

11. The composition according to claim 10, wherein the mixture of monomers comprises at least one semi-hydrophobic surfactant vinyl monomer chosen from a compound of the following formulae:
CH₂═CH—O(CH₂)ₐO(C₃H₆O)_{b}(C₂H₄O)CH and
CH₂═CHCH₂O(G₃H₆O)_{d}(C₂H₄O)ₑH;
wherein:
a is an integer ranging from 2 to 4;
b is an integer ranging from 1 to 10;
c is an integer ranging from 5 to 50;
d is an integer ranging from 1 to 10; and
e is an integer ranging from 5 to 50.

12. The composition according to claim 10, wherein the at least one semi-hydrophobic surfactant vinyl monomer is present in an amount ranging from 0% to 25% by weight, relative to the total weight of the mixture of monomers.

13. The composition according to claim 1, wherein the at least one hydroxylated nonionic vinyl monomer e) is chosen from C₁-C₆ hydroxyalkyl (meth)acrylates and (C₁-C₄ hydroxyalkyl)(meth)acrylamides.

14. The composition according to claim 13, wherein the at least one hydroxylated nonionic vinyl monomer is 2-hydroxyethyl methacrylate.

15. The composition according to claim 1, wherein the at least one hydroxylated nonionic vinyl monomer is present in an amount ranging from 0.01% to 10% by weight, relative to the weight of the mixture of monomers.

16. The composition according to claim 1, wherein the mixture of monomers comprises, relative to the total weight of the mixture of monomers:
a) from 20% to 60% by weight of the at least one vinyl monomer substituted with at least one amino group,
b) from 20% to 70% by weight of the at least one hydrophobic nonionic vinyl monomer,
c) from 0.01% to 15% by weight of the at least one associative vinyl monomer,
d) from 0.1% to 10% by weight of the at least one semi-hydrophobic surfactant vinyl monomer,
e) from 0.01% to 10% by weight of the at least one hydroxylated nonionic vinyl monomer,
f) from 0.001% to 5% by weight of the at least one crosslinking monomer,
g) from 0.001% to 10% by weight of the at least one chain-transfer agent, and
h) from 0 to 2% by weight of the at least one polymeric stabilizer.

17. The composition according to claim 1, wherein the mixture of monomers comprises:
a di(C₁-C₄ alkyl)amino(C₁-C₆ alkyl)methacrylate,
at least one (meth)acrylic acid C₁-C₃₀ alkyl ester,
a polyethoxylated C₁₀-C₃₀ alkyl methacrylate with from 20 mol to 30 mol of ethylene oxide,
a polyethylene glycol/polypropylene glycol 30/5 alkyl ether,
a hydroxy(C₂-C₆ alkyl)methacrylate, and
an ethylene glycol dimethacrylate.

18. The composition according to claim 1, wherein the at least one cationic polymer is present at a concentration ranging from 0.01% to 10% by weight, relative to the total weight of the composition.

19. The composition according to claim 1, wherein the at least one anionic associative polymer is chosen from copolymers of (meth)acrylic acid and of allylic ethers of C₈₋₃₀ fatty alcohols, copolymers of unsaturated carboxylic acids and of unsaturated C₁₀₋₃₀ alkyl carboxylates, terpolymers of maleic anhydride/C₃₀₋₃₈ α-olefin/alkyl maleate, acrylic terpolymers obtained (a) from an α,β-ethylenically unsaturated carboxylic acid, (b) from a non-surfactant α,β-ethylenically unsaturated monomer other than (a), and (c) from a nonionic surfactant monomer obtained by reacting an ethylenically unsaturated monoisocyanate and a monohydric surfactant, copolymers of α,β-monoethylenically unsaturated carboxylic acids, of an ester of an α,β-monoethylenically unsaturated carboxylic acid and of an oxyalkylenated fatty alcohol, and amphiphilic polymers comprising at least one ethylenically unsaturated monomer containing a sulfonic group, the polymer comprising at least one hydrophobic part.

20. The composition according to claim 1, wherein the at least one anionic associative polymer is an acrylic terpolymer of an α,β-ethylenically unsaturated carboxylic acid, of a nonionic urethane monomer which is the product of a reaction of a monohydric nonionic amphiphilic compound with a monoethylenically unsaturated isocyanate.

21. The composition according to claim 20, wherein the acrylic terpolymer comprises, relative to the total weight of the terpolymer:
(a) from 20% to 70% by weight of an α,β-ethylenically unsaturated carboxylic acid,
(b) from 20% to 80% by weight of a non-surfactant ethylenically unsaturated monomer other than (a), and
(c) from 0.5% to 60% by weight of a nonionic urethane monomer which is the product of a reaction of a monohydric nonionic amphiphilic compound with a monoethylenically unsaturated isocyanate.

22. The composition according to claim 1, wherein the at least one anionic associative polymer comprises at least one ethylenically unsaturated monomer containing a sulfonic group.

23. The composition according to claim 1, wherein the at least one anionic associative polymer is present in an amount ranging from 0.01% to 20% by weight, relative to the total weight of the composition.

24. The composition according to claim 1, wherein the composition further comprises at least one surfactant chosen from anionic, amphoteric, nonionic, and cationic surfactants.

25. The composition according to claim 24, wherein the at least one surfactant is present in an amount ranging from 0.01% to 50% by weight, relative to the total weight of the composition.

26. The composition according to claim 24, wherein the at least one surfactant is present in an amount of at least 4% by weight, relative to the total weight of the composition.

27. The composition according to claim 1, wherein the composition further comprises at least one conditioning agent.

28. The composition according to claim 27, wherein the at least one conditioning agent is chosen from poly-α-olefins, fluoro oils, fluoro waxes, fluoro gums, carboxylic acid esters, silicones, cationic polymers other than those of the present disclosure, mineral, plant, and animal oils, ceramides, and pseudoceramides.

29. The composition according to claim 28, wherein the at least one conditioning agent is a silicone.

30. The composition according to claim 29, wherein the at least one conditioning agent is a cationic polymer.

31. The composition according to claim 29, wherein the at least one conditioning agent is present in an amount ranging from 0.001% to 10% by weight, relative to the total weight of the composition.

32. The composition according to claim 1, wherein the composition is in the form of a foaming detergent composition or a conditioner.

33. The composition according to claim 32, wherein the foaming detergent composition is a shampoo, shower gel, makeup-removing product, or bubble bath.

34. The composition according to claim 32, wherein the conditioner is a rinse-out or leave-in conditioner.

35. The composition according to claim 1, wherein the composition is chosen from compositions for permanent-waving, hair-straightening, dyeing, and bleaching, or compositions to be applied before or after a dyeing, bleaching, permanent-waving, or hair-straightening operation, and compositions to be applied between steps of a permanent-waving or hair-straightening operation.

36. A method of treating keratin materials comprising
1) applying a cosmetic composition comprising, in a cosmetically acceptable medium:
(i) at least one cationic polymer which is produced by polymerization of a mixture of monomers comprising:
a) at least one vinyl monomer substituted with at least one amino group,
b) at least one hydrophobic nonionic vinyl monomer, chosen from a compound of formula (I) and (II):
CH₂═C(X)Z, (I)
CH₂═CH—OC(O)R; (II)
wherein:
X is a hydrogen atom or a methyl group;
Z is chosen from —C(O)OR¹, —C(O)NH₂, —C(O)NHR¹, —C(O)N(R¹)₂, —C₆H₅—C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—NH—CH₂CH₂-urea, —Si(R)₃, —C(O)O(CH₂)ₓSi(R)₃, —C(O)NH(CH₂)ₓSi(R)₃, and —(CH₂)ₓSi(R)₃;
x is an integer ranging from 1 to 6;
each R is independently a C₁-C₃₀ alkyl group; and
each R¹ is independently chosen from a C₁-C₃₀ alkyl group, a hydroxylated C₂-C₃₀ alkyl group, and a halogenated C₁-C₃₀ alkyl group; and
c) at least one associative vinyl monomer, and
e) at least one hydroxylated nonionic vinyl monomer; and
(ii) at least one anionic associative polymer;
and
2) optionally rinsing with water.

37. The method according to claim 36, wherein the keratinous material is hair.

38. The method according to claim 36, wherein optionally rinsing with water follows a leave-in time.
